SEQUENCE LISTING (35 sequences)

SEQ ID NO: 1 - protein, 914 aa, Bacillus subtilis
MSDQFNSREARRKANSKSSPSPKKGKKRKKGGLFKKTLFTLLILFVLGVVGGAVTFAVMVSDAPSLDESKLKTPYSSTIYDKNGKEIAEVGAEKRTYVSIDEIPDVVKEAFIATEDARFYEHHGIDPVRIGGALVANFKDGFGAEGGSTITQQVVKNSLLSHQKTLKRKVQEVWLSIQLERNYSKDEILEMYLNRIYFSPRAYGIGKAAEEFFGVTDLSKLTVEQAATLAGMPQSPTAYNPVKNPDKAEKRRNIVLSLMKKQGFISDSQYNKAKKVAVKDEGVVSQKEYEKASTNKYSAFVEEVMKEIDEKSDVDPSADGLKIYTTLDTKAQDKLDELMDGDTVGFTEGMQGGVTLLDTKNGEVRAIGAGRNQPVGGFNYATQTKAQPGSTIKPILDYGPVIENKKWSTYEQIDDSAYTYSNGKPIRDWDRKYLGPISMRYALAQSRNIPALKAFQAVGKDTAVDFANGLGLGLTKDNVTEAYSIGGFGGNDGVSPLTMAGAYSAFGNNGTYNEPHFVKSIEFNDGTKLDLTPKSKSAMSDYTAFMITDMLKTAVKTGTGQLAQVPGVEVAGKTGTTNFDDNEVKRYNIASGGARDSWFVGYTPQYTAAVWTGMGENEAGKKSLSAEEQKVAKRIFAQLIADVDDGSGSFEKPDSVVEATVEKGSNPAKLAGPNTPSDKKLTEYFVKGTAPSTVSKTYEKEEKEETAKLSGLNVKYDKDNQSLTLSWNYDGDATFAVKQSVDGGSYSEIQNSSAKEAVISGVQPGSVYKFEVTAVSDDGKSTASTSYEVPKAEDDEDKKDQQQTDDEKQDDEKTQDDTQTDDSQKDDGQTDQDQTDDSTNDQDKKQDNTNTNPSDNNNQDQSNDNDNDNSNNQDTSDGDSNSGKNDSTGSDTNKNKTDTSNKTQTNSSSIEKTN

SEQ ID NO: 2 - protein, 895 aa, Bacillus licheniformis
MSDQFNSRQERRKAQQGKSRSNTHSKPKKKKKAGLFKKILLSILIIGVIGLIAGGVTFAVMVADSPSLDEAKLKTPYSSTIYDKNGKEIAEIGSEKRTYVSIKDIPDSVKNAFLATEDARFYDHHGVDPIRIGGALLANFEGGFGSEGGSTITQQVVKNSLLSHEKTLKRKVQEVWLSFQLERKYSKDEILEMYLNRIYFSPQAYGVGKAAEQFYGVTDLNDLTVEQAATLAGMPQSPNNYNPIKHPEKAEKRRNVVLSLMNKHGFISDAEYNKAKKVAVTKGLVSPKEYAKTTSNKYSAFIEQAVAEVKEKANVTPGTDGLKIYTTIDTDAQDYVDELMDGDSIQYTEKMQAGLTLLDTKTGEIRALGGGRDRKAGDFNYAVDTKRQPGSTIKPILDYGPVIENKKWSTYEQIKDEAYTYSTGDPINNYDRSYRGWISMREALVDSRNIPALKAFQAAGKDNIVKFAGNLGLNINSDDLVEAYAIGGFGNGVSPLQMAGAYSAFGNNGYYNEPHTVTAVEFNDGTKLDLTPESKAAMSDYTAFMITDMLKSAVQRGTGTAAQVPGVTVAGKTGTTNFTEDDIRKHGINRNGARDSWFVGYTPQYTAAVWTGKEGFNSLSQSEQQVAKLLFKKLIAKVDNGSGSFEKPDSVVEATVLKGSNPPVLASSNTPSDKKVTEYFVKGTQPTTVSKKYEEKENADKPSGLSAEYDEASKSIKLSWSYSGDGDVSFKVKQSVDGGGYSEIQNSSAKEAVIPNAKEGSVYRFQVTAVTEDGESDPASVTLKVNAADEDESKTDDEKKDDDEKKQDADDDQDKDKQNNGNSNQPNDQNPGNSDQNNQNPGNSDQNNGRKDDDADDQDNDQNKDKDKDKNKNPDQGANTGDQQTNSNGQSTGTN

SEQ ID NO: 3 - protein, 926 aa, Bacillus amyloliquefaciens
MSDQFKSREERRKARQTKSPKPSSPKKGKKQRKGGLFKKIVLSLVILFVLGIVGGAAAFAVLVSGAPSLDEAKLKTPYSSTIYDKNGKEIAEVGGEKRTYVPIKDIPDVVKEAFIATEDARFYKHHGIDPIRIGGALVANFTDGFGSEGGSTITQQVVKNSLLTHQKTIKRKAQEVWLSLQLERHYSKDEILEMYLNKIYFSPRAYGVGKAAEDFFGVKDLSKLTVEQAATLAGMPQSPTGYNPVKNPKASEKRRNIVLSLMEKQGFISNSEYQKAKKVSMKDEGVVSQKEYDKKDTNKYSAFVEEVMKEIETKEKKDVDISTDGLKIYTTLDTKAQDYLDQLMNGDTVGFTQGMQGGVTLLDTTNGEIRAIGAGRNRTAGGFNYATDTQRQPGSTIKPILDYGPVIENKKWSTYEQIDDSAYTYSDGTPIHDYDNKYRQMITMREALSDSRNIPALKAFQAAGKDNAVDFANDLGLGLNKDSVTESYSIGGFGADNPGVSPLTMAGAYSAFGNNGTYNEPHAVTSIEFNDGTKLDLTPKPKSAMSDYTAFMITDMLKTVVQSGTGKLAQVPNVEVAGKTGTTNFTKEDIQKYNIAPGGVPDSWFVGYTPQYTAAVWTGVESGNKAGKKSLSYADQQVAKRIFQKLISYVDDGKGSFKQPDSVVKETVEKGSNPAALAGPNTPDDKKVTEYFVKGTEPTAVSKTYKKEKANKPTGLQVKYDKDKKSLNLSWDYDGDATFNVKQSVDGGSYSDIQNSSAKEAVVSDVTPGSTYSFQVTAENDDGKSDTASASFKVPKDEEDNADKDKATDDEQKPDDKDKDKTQTDDSKTDDNQTDTSDGSDQTPTDDPNKNPDQSNQDQTDNNNPNQPGNGTDNNGQQDNSSDNSSDTGSDGSKNPDSGQKDKNQDSSSTVNGQSNSSTIQSNTAN

SEQ ID NO: 4 - protein, 948 aa, Bacillus megaterium
MSDNYRSREERRRAMNDNKPEGKSQGKPKKKKKGGLFRKIVAAVLLIGIIGLIAGVGTFFAMISDAPKVDDSVLKNSFSSKVYANDGKTVVKEIGAQKRTYVQYDEIPQVVKNAFIATEDVRFYKHHGVDFYRIGGALMANFKNGFGAEGGSTITQQVVKNSFLSPKKTVKRKVQEMWLAYQLERKYSKQQILEMYLNKIYFASGEVYGIERAAEKFYGVKSVKDLTLDEAAMLAGLPKAPTTYNPVTNPENATKRRNTVLNLMAKNGFITQAEADKAKQVDVQAHLAKQTESTSKYDVFIDQVIEEVKKKTDADPFSAGLEIYTTLDTNAQDYVDDVMNNKVVNFPNDKFQAGLVVVDNETGGIQAIGGGRNRVSGGLNFATNMRRQPGSTIKPILDYGPAIENLKWSTGQPLKDEEYHYSNGTPIRNFGRNYKGWVSAREALGRSLNIPALKAFQATGADKAKEFATGLGLKLDEEDGEAYSESYSIGGFRTGVTPLQMAGAYSAFANEGMYNAPHTVTKVKFPDGTEIDLTPKSKRAMQDYTAFMMTDMMKSVVNESYGTARAVRTPGLEIAGKTGTSNFSAAEKQRYNIPDAGEKDVWFNGFTPNYTISVWTGYETAKDGYILSPNETGMAKNIFKHVITHISEGKPKGSFKQPDSVVERSVEKPTGKDLKRPSGSTPSSLITKEYFVRGTAPTKVSQTFVKKEKEEKKEEKEEKKEDKKEKKDNKNVKPKITASYSGGAISVNWSASNAAGDTTFTVTMSSPNGSEQLASGAGAGGKTISNPAPGATYSFTVTTNTGESASSSVKVPDSSDQATEDDSTKPEDDGTTDDNQKEDNSNTDQNNGSDQGDDSSTQPDDNQNSNNGSNNGSNNGGNNSGNTDNNNNDSGNANDGNSNNNNNNGGTTTPPADNGGSDSGSQTPPTTPATPSTPSTPATPQQEPTTEN

SEQ ID NO: 5 - protein, 891 aa, Geobacillus stearothermophilus
MSGEYRSRVERKQAAKQTKQKKAKKKRGRSWLKAIAIAIVLMIALGAIGGVAAFAYFVKDAPPLDEAKIKDPLSSTVYDMSGKKVAELGGYKRTYISYKDVPKVLENAVLATEDARFYEHHGIDIVRLAGAVIANIREGFGAEGGSTITQQVVKMTFLSPKKTLERKAQEAWLALQLEQKYSKHEILEMYLNKIYYSDGIYGVARAAEYYFGKTNLKELTLPEAALLAGMPQSPNRYNPYVHPEAAKKRRDTVLSLMVKHGFISAEEAEKAKQVPIKSMLTERKKPQNSVPYDAFIDEVIKEVTDKANVNVFEDGLKIYTTLDQEAQSYVEKLLNSDQRFTAKKDLQSGIALVDTKTGAIRALGGGRHRDNIEFGFNYAIQPVGQPGSTIKPILDYGPAIEYLKWSTAHILVDEPYTYSDGTPIRNAGGRYAGPVTVRDALTWSRNIPALKTFQAVGKERAREFANRLGMGFDEVYEPYAIGGVSRYVSPLQMAGAYAAFGNNGVYTKPYAVTKIVFPDGTEMDLKPKPKRVMHDYTAYMITDMLKSVIRSGTGQSAYVPGLELAGKTGTTNYGKDGAKFGLKRDDVPDSWIVGYTPNYTAAIWVGFSKKSETATLGDREQRIPKLLLKDLMSHLDDGSGEFQRPDSVVKLPIKKGTNPPKLAGKYTPESDITYELFVRGTEPTEVAKDEPEELPAVKGLAASYDQAANIISVSWSYDEKTDDTLFEVRIKDEQGHTETVTTKDLGVTVTNVAPGASYTIAVYAKEGERMSKPAITSIRIPGGEQPTAPGNEANDDGNNHGAGNGGTNGDNGDDHGGNGNNQNNNNGGNSGNNGNGNEDRDDGGEEDNGGEDNGQGDGGATTPTTPTTPTTPTNPQSGNSGNAGMRNGSRQ

SEQ ID NO: 6 - protein, 721 aa, Bacillus subtilis
MSDQFNSREARRKANSKSSPSPKKGKKRKKGGLFKKTLFTLLILFVLGVVGGAVTFAVMVSDAPSLDESKLKTPYSSTIYDKNGKEIAEVGAEKRTYVSIDEIPDVVKEAFIATEDARFYEHHGIDPVRIGGALVANFKDGFGAEGGSTITQQVVKNSLLSHQKTLKRKVQEVWLSIQLERNYSKDEILEMYLNRIYFSPRAYGIGKAAEEFFGVTDLSKLTVEQAATLAGMPQSPTAYNPVKNPDKAEKRRNIVLSLMKKQGFISDSQYNKAKKVAVKDEGVVSQKEYEKASTNKYSAFVEEVMKEIDEKSDVDPSADGLKIYTTLDTKAQDKLDELMDGDTVGFTEGMQGGVTLLDTKNGEVRAIGAGRNQPVGGFNYATQTKAQPGSTIKPILDYGPVIENKKWSTYEQIDDSAYTYSNGKPIRDWDRKYLGPISMRYALAQSRNIPALKAFQAVGKDTAVDFANGLGLGLTKDNVTEAYSIGGFGGNDGVSPLTMAGAYSAFGNNGTYNEPHFVKSIEFNDGTKLDLTPKSKSAMSDYTAFMITDMLKTAVKTGTGQLAQVPGVEVAGKTGTTNFDDNEVKRYNIASGGARDSWFVGYTPQYTAAVWTGMGENEAGKKSLSAEEQKVAKRIFAQLIADVDDGSGSFEKPDSVVEATVEKGSNPAKLAGPNTPSDKKLTEYFVKGTAPSTVSKTYEKEEKEETAKLSGLNVKYDKDNQ

SEQ ID NO: 7 - protein, 699 aa, Bacillus licheniformis
MSDQFNSRQERRKAQQGKSRSNTHSKPKKKKKAGLFKKILLSILIIGVIGLIAGGVTFAVMVADSPSLDEAKLKTPYSSTIYDKNGKEIAEIGSEKRTYVSIKDIPDSVKNAFLATEDARFYDHHGVDPIRIGGALLANFEGGFGSEGGSTITQQVVKNSLLSHEKTLKRKVQEVWLSFQLERKYSKDEILEMYLNRIYFSPQAYGVGKAAEQFYGVTDLNDLTVEQAATLAGMPQSPNNYNPIKHPEKAEKRRNVVLSLMNKHGFISDAEYNKAKKVAVTKGLVSPKEYAKTTSNKYSAFIEQAVAEVKEKANVTPGTDGLKIYTTIDTDAQDYVDELMDGDSIQYTEKMQAGLTLLDTKTGEIRALGGGRDRKAGDFNYAVDTKRQPGSTIKPILDYGPVIENKKWSTYEQIKDEAYTYSTGDPINNYDRSYRGWISMREALVDSRNIPALKAFQAAGKDNIVKFAGNLGLNINSDDLVEAYAIGGFGNGVSPLQMAGAYSAFGNNGYYNEPHTVTAVEFNDGTKLDLTPESKAAMSDYTAFMITDMLKSAVQRGTGTAAQVPGVTVAGKTGTTNFTEDDIRKHGINRNGARDSWFVGYTPQYTAAVWTGKEGFNSLSQSEQQVAKLLFKKLIAKVDNGSGSFEKPDSVVEATVLKGSNPPVLASSNTPSDKKVTEYFVKGTQPTTVSKKYEEKENA

SEQ ID NO: 8 - protein, 710 aa, Bacillus amyloliquefaciens
MSDQFKSREERRKARQTKSPKPSSPKKGKKQRKGGLFKKIVLSLVILFVLGIVGGAAAFAVLVSGAPSLDEAKLKTPYSSTIYDKNGKEIAEVGGEKRTYVPIKDIPDVVKEAFIATEDARFYKHHGIDPIRIGGALVANFTDGFGSEGGSTITQQVVKNSLLTHQKTIKRKAQEVWLSLQLERHYSKDEILEMYLNKIYFSPRAYGVGKAAEDFFGVKDLSKLTVEQAATLAGMPQSPTGYNPVKNPKASEKRRNIVLSLMEKQGFISNSEYQKAKKVSMKDEGVVSQKEYDKKDTNKYSAFVEEVMKEIETKEKKDVDISTDGLKIYTTLDTKAQDYLDQLMNGDTVGFTQGMQGGVTLLDTTNGEIRAIGAGRNRTAGGFNYATDTQRQPGSTIKPILDYGPVIENKKWSTYEQIDDSAYTYSDGTPIHDYDNKYRQMITMREALSDSRNIPALKAFQAAGKDNAVDFANDLGLGLNKDSVTESYSIGGFGADNPGVSPLTMAGAYSAFGNNGTYNEPHAVTSIEFNDGTKLDLTPKPKSAMSDYTAFMITDMLKTVVQSGTGKLAQVPNVEVAGKTGTTNFTKEDIQKYNIAPGGVPDSWFVGYTPQYTAAVWTGVESGNKAGKKSLSYADQQVAKRIFQKLISYVDDGKGSFKQPDSVVKETVEKGSNPAALAGPNTPDDKKVTEYFVKGTEPTAVSKTYKKEKA

SEQ ID NO: 9 - protein, 704 aa, Bacillus megaterium
MSDNYRSREERRRAMNDNKPEGKSQGKPKKKKKGGLFRKIVAAVLLIGIIGLIAGVGTFFAMISDAPKVDDSVLKNSFSSKVYANDGKTVVKEIGAQKRTYVQYDEIPQVVKNAFIATEDVRFYKHHGVDFYRIGGALMANFKNGFGAEGGSTITQQVVKNSFLSPKKTVKRKVQEMWLAYQLERKYSKQQILEMYLNKIYFASGEVYGIERAAEKFYGVKSVKDLTLDEAAMLAGLPKAPTTYNPVTNPENATKRRNTVLNLMAKNGFITQAEADKAKQVDVQAHLAKQTESTSKYDVFIDQVIEEVKKKTDADPFSAGLEIYTTLDTNAQDYVDDVMNNKVVNFPNDKFQAGLVVVDNETGGIQAIGGGRNRVSGGLNFATNMRRQPGSTIKPILDYGPAIENLKWSTGQPLKDEEYHYSNGTPIRNFGRNYKGWVSAREALGRSLNIPALKAFQATGADKAKEFATGLGLKLDEEDGEAYSESYSIGGFRTGVTPLQMAGAYSAFANEGMYNAPHTVTKVKFPDGTEIDLTPKSKRAMQDYTAFMMTDMMKSVVNESYGTARAVRTPGLEIAGKTGTSNFSAAEKQRYNIPDAGEKDVWFNGFTPNYTISVWTGYETAKDGYILSPNETGMAKNIFKHVITHISEGKPKGSFKQPDSVVERSVEKPTGKDLKRPSGSTPSSLITKEYFVRGTAPTKVSQTF

SEQ ID NO: 10 - protein, 694 aa, Geobacillus stearothermophilus
MSGEYRSRVERKQAAKQTKQKKAKKKRGRSWLKAIAIAIVLMIALGAIGGVAAFAYFVKDAPPLDEAKIKDPLSSTVYDMSGKKVAELGGYKRTYISYKDVPKVLENAVLATEDARFYEHHGIDIVRLAGAVIANIREGFGAEGGSTITQQVVKMTFLSPKKTLERKAQEAWLALQLEQKYSKHEILEMYLNKIYYSDGIYGVARAAEYYFGKTNLKELTLPEAALLAGMPQSPNRYNPYVHPEAAKKRRDTVLSLMVKHGFISAEEAEKAKQVPIKSMLTERKKPQNSVPYDAFIDEVIKEVTDKANVNVFEDGLKIYTTLDQEAQSYVEKLLNSDQRFTAKKDLQSGIALVDTKTGAIRALGGGRHRDNIEFGFNYAIQPVGQPGSTIKPILDYGPAIEYLKWSTAHILVDEPYTYSDGTPIRNAGGRYAGPVTVRDALTWSRNIPALKTFQAVGKERAREFANRLGMGFDEVYEPYAIGGVSRYVSPLQMAGAYAAFGNNGVYTKPYAVTKIVFPDGTEMDLKPKPKRVMHDYTAYMITDMLKSVIRSGTGQSAYVPGLELAGKTGTTNYGKDGAKFGLKRDDVPDSWIVGYTPNYTAAIWVGFSKKSETATLGDREQRIPKLLLKDLMSHLDDGSGEFQRPDSVVKLPIKKGTNPPKLAGKYTPESDITYELFVRGTEPTEVAKDEPEEL

SEQ ID NO: 11 - DNA, 2745 bp, synthetic construct
  source 1..2745: mol_type = other DNA
atgtcagatcaatttaacagccgtgaagctcgacgaaaagcgaacagcaaatcgagtccttcaccgaaaaaaggcaagaaacgaaaaaagggcggattgtttaaaaagacccttttcactttactcattctgtttgttttaggcgtagtcggcggtgctgttacatttgccgtcatggtttctgatgcgccgtcccttgatgaaagcaaattgaagacgccttattcatcaacgatttatgataaaaatggaaaagagatcgccgaagtcggcgccgaaaaacggacctacgtctcgatagatgaaattcccgatgttgtaaaagaagcctttatcgcgacagaagacgctcgtttttatgagcaccacgggattgaccctgtccgtatcggcggtgcattagttgccaactttaaagacggcttcggggctgaaggcggaagtacgatcacccagcaggtcgtcaaaaactcccttctttcacatcagaagacgttgaaacggaaggtgcaggaagtatggctttcgattcagctggagcgcaattactctaaagatgaaattttagaaatgtatttaaaccggatttatttttctcctagagcatacggaatcggaaaagcggcggaagaatttttcggcgttactgatttaagcaaattgactgtcgaacaggctgcgacgcttgcaggaatgccgcagagcccaacagcgtacaacccggtcaaaaacccggataaagcggaaaaacgacggaacatcgtactcagtctgatgaaaaagcaaggatttatttctgattctcaatataacaaagcgaaaaaagtggcagtgaaagacgaaggcgtcgtgtcacagaaggaatatgaaaaagcaagtacaaacaaatacagcgcatttgttgaagaggttatgaaggaaattgatgaaaaatctgatgtcgatccatctgctgacggattaaagatctatacgacattagatacaaaagcacaagataaactagatgaattaatggacggagacaccgtcggatttactgaaggcatgcagggcggtgtgacgcttctcgataccaaaaacggagaagtccgagcgattggtgcgggacgcaatcagcctgtcggaggctttaactatgctactcaaactaaggcacagcctggttcgaccataaaaccgattttggactacggaccagttattgaaaataagaaatggtccacatacgaacaaattgatgactcagcttatacgtattctaacggaaaaccgattcgtgattgggaccgcaaatatctagggcctatctcaatgcgttacgctctggcccaatcaagaaatatacctgctttaaaagcattccaggcagtcggtaaagatactgctgtagactttgcaaatggactcgggcttggtttaacaaaagataatgtaacagaggcctattctattggcggtttcggtgggaacgatggtgtttctcctctgacaatggcaggtgcatacagcgcgtttggaaataacggaacgtataatgaaccgcattttgtaaaatctatcgaatttaacgatggcacgaagcttgacttaacaccaaaatcaaaatcagccatgagtgattatactgcgtttatgattacagatatgctgaaaacagctgtgaagacaggtactggacagcttgcacaagtacctggtgtagaagttgcaggaaaaacaggaacgactaactttgatgataatgaagtcaaaaggtacaatatcgctagcggtggcgcccgagattcttggttcgttggctacacaccgcaatatacagctgccgtctggacgggaatgggagaaaacgaggctggaaagaaatcactttcagctgaagagcaaaaagttgcaaagcgcatcttcgcccagctcatcgccgatgtcgatgacggaagcggatcatttgagaagcctgacagcgtagtggaagccaccgtagaaaaaggttctaatccggcaaaactggcagggccaaatacgccaagcgataagaagctcacagagtactttgttaaaggcacagctccttctactgtttctaaaacatatgagaaagaagaaaaagaggaaacagctaaactgtctggtttaaacgtgaaatacgataaagacaatcaatctttaacgttaagctggaattacgacggagatgcgacctttgctgttaagcaatctgttgacggcggcagctactcggaaattcaaaacagctctgcaaaagaggcagttatttcgggtgtgcagccaggatctgtatacaaattcgaagtaacagccgtcagtgatgatggcaaaagtacagcttccacatcttatgaggtgccaaaagctgaagacgatgaagataaaaaagaccagcaacaaacggatgatgaaaaacaagatgatgagaagactcaggatgatacacaaactgatgattctcaaaaagatgacggtcagacggatcaagatcagacagatgattcaacaaacgatcaagacaaaaaacaagacaatacgaacaccaatccgtccgacaacaataaccaagaccaatcaaacgataatgataacgacaacagtaacaaccaggatacgtcggatggtgattcgaactctggtaagaatgattcaacaggttctgacacgaataaaaataaaacagacacatctaataaaacacaaacaaactcatcatccattgaaaaaacaaattaa

SEQ ID NO: 12 - DNA, 2688 bp, synthetic construct
  source 1..2688: mol_type = other DNA
atgtcagatcaatttaacagccgccaggaacggcgaaaagcccagcagggcaaaagcaggtctaatactcattctaaacccaagaaaaagaaaaaagcaggattgtttaagaaaattcttttatccattttaatcatcggtgttatcggcttgattgccgggggcgtcacttttgcggtgatggtagcggactcgccgtcccttgatgaagcaaaactgaagacaccgtactcttctaccatctatgacaaaaacggaaaagaaatcgccgaaatcggttcggaaaaacggacgtacgtgtcgattaaagacatcccggattccgtcaaaaacgcatttcttgcaacggaagacgcccgtttctatgaccaccacggcgtcgacccgatccggatcggaggagcgcttctggcaaacttcgaaggcggatttggttctgaaggcggaagtacgatcacacagcaggtcgtcaaaaactcccttctgtcacatgaaaaaacgctgaaacggaaagtccaggaagtctggctgtctttccagctggaaagaaaatactcaaaagacgaaattcttgaaatgtatttaaatcggatttacttctcgccgcaggcctacggtgtcggaaaagcggctgagcagttctacggggttacagatctaaacgacttaacggtcgagcaggccgcaacgcttgccggcatgccgcaaagcccgaataactacaacccgatcaaacatccggaaaaagccgagaaacggcgcaatgtcgtgctgagcctgatgaataaacacggctttatctctgacgcagagtacaacaaagcgaaaaaagtagccgtcacaaaaggccttgtatctccgaaagagtacgcaaaaacaacgtccaacaaatacagcgcgtttatcgagcaggccgtcgccgaagtgaaagaaaaagcgaacgtcactccgggaacggacggactgaagatttacacgacgattgatacagacgcacaggattatgtggatgagctgatggacggcgattctattcagtataccgaaaaaatgcaggccggcctcaccctgctcgatacgaaaaccggagagatccgcgcgctcggcggcggacgcgaccggaaagcaggcgactttaactatgcagtagatactaaacgtcaacccggttcaacgattaagccaatccttgactacggccctgtcattgagaacaagaaatggtcgacgtatgaacagattaaagatgaagcatacacgtattcaactggagatccgattaacaactatgacagaagctacagaggctggatatcgatgagagaggcgctggtcgattctcgaaacatcccggctttaaaagcgttccaggcagccggcaaagacaatattgtaaaatttgcaggcaaccttggccttaacataaattcagatgacttagtggaagcttatgcaatcggcggcttcggaaatggtgtgtcgcctctgcaaatggccggagcgtacagcgccttcggcaacaacggctactacaatgagccgcatactgttaccgcggtggaattcaatgacggcactaagcttgatttgacgcctgaatcaaaggctgccatgagcgattataccgcatttatgataacggatatgctgaaaagcgctgttcagcgaggaacaggaacagcggctcaagtccccggcgtcactgtagcaggtaaaacgggtacgacgaactttaccgaggacgacattagaaaacacggcataaatcgaaacggagcgagagattcatggttcgtcggctatacgccgcaatataccgcagcggtctggaccggtaaagaaggctttaattcgctgagccagagcgaacagcaagtcgccaagctcctattcaaaaaactgattgccaaggtggacaacggcagcggctcatttgaaaagcctgacagcgtcgttgaagcgaccgttctaaaaggctcgaacccgccggtgctcgccagcagcaacacgcctagcgataaaaaagtcactgagtactttgtaaaaggcacacagccgacgaccgtttcgaaaaaatatgaagaaaaagaaaatgccgacaagccgtcaggtttgagcgcagagtatgatgaagcatcaaagagcattaagctttcatggtcatattccggagacggtgatgtatcctttaaagtcaaacagtctgttgacggaggcggatacagcgaaattcaaaacagcagtgcgaaagaagccgtcataccgaatgcaaaagaaggatccgtttacagattccaagtcactgccgttaccgaagatggtgaaagcgaccctgcttctgtaacgcttaaagtcaacgcggctgatgaggatgagtcaaaaacggacgatgaaaagaaagacgatgacgagaagaagcaggatgctgatgatgatcaggacaaagacaaacaaaataacggcaattcaaatcagccgaatgatcaaaatccaggaaattccgatcaaaacaatcaaaatccgggaaattcagaccaaaacaacggcagaaaagatgatgacgccgatgatcaagacaacgatcaaaataaagataaagataaagacaaaaacaaaaacccggatcaaggcgccaacacaggtgatcaacaaacaaattccaacggtcaatcaaccggcaccaattga

SEQ ID NO: 13 - DNA, 2781 bp, synthetic construct
  source 1..2781: mol_type = other DNA
atgtcagatcaatttaagagccgtgaagaacgccgaaaggcaaggcagacgaaaagccccaagccttcttcaccgaaaaaaggcaagaaacagcgtaaaggcggtttattcaaaaagattgtactgtccttggttattctgtttgtcctcggcatagtcggcggagctgcggcatttgccgttttagtttccggtgcgccgtctttggacgaagccaagctgaaaacgccttactcttcaacaatttacgataaaaacggcaaagaaatcgcggaagtgggcggcgaaaaacgaacctacgtcccgattaaggatatacccgatgttgtgaaggaagcttttatcgcaacagaggacgcccgtttttataagcaccacggaatagacccgatccggatcggaggcgccttggtcgccaactttacggatggtttcggttcagaaggcggaagcacaatcacccagcaggttgtgaaaaactcgcttctgacccaccagaaaacgattaaacggaaagctcaggaagtatggctttcactgcagctggagcgccattattctaaagatgaaattttagaaatgtatttaaacaaaatttatttctcaccgcgcgcctacggcgtcggtaaagccgctgaagactttttcggcgtgaaagatttgagcaagcttacggttgaacaggcagcaacccttgccggaatgccgcaaagtccgaccggatacaacccggttaaaaatccgaaggcttctgaaaaacgccggaacatcgttctcagcttaatggaaaaacaaggatttatctcaaattctgaataccaaaaagcaaagaaagtgtccatgaaagatgaaggcgtcgtcagccaaaaggaatacgataaaaaggacacaaataaatacagcgcttttgtcgaagaagttatgaaagaaattgagacaaaagaaaagaaagacgtcgatatttcaactgacggattaaaaatttacacgactcttgatacgaaagcacaagattatcttgatcaattgatgaacggcgacacagtcggctttacccaaggcatgcagggcggcgtcaccctccttgatacgacaaacggagaaatccgcgccatcggagcaggacgaaacagaactgccgggggatttaactacgcgacagacactcaacgccagccaggttcaacgataaaaccgattcttgattacggtccggtcattgaaaataaaaaatggtcgacgtatgagcagattgatgactcagcctatacgtactctgacggcactccgattcatgactatgacaataaatacagacaaatgataacaatgcgagaggctctctcagactcacgtaatattcctgctctgaaggcattccaagcagcaggtaaagataatgccgtcgacttcgcaaacgacttaggactaggattaaacaaagacagtgtaacagaatcttattcgatcggcggttttggagctgataacccgggcgtttccccgctgactatggctggtgcctacagcgcttttggaaataacggaacgtacaatgaaccgcacgcggtcacatcgattgaatttaatgacggaactaagcttgatttaacaccgaaaccgaagagtgctatgagcgattacacggcattcatgattaccgatatgctaaaaacagtggtgcagtcaggaacgggtaaactggctcaagtaccgaacgtggaagtcgccggtaaaacgggtacgacaaactttactaaagaggatattcaaaaatataacattgctccaggcggagtaccggattcatggtttgtcggctacacccctcaatatacagcggcggtttggaccggtgttgagtctggtaacaaagcaggtaaaaaatcattatcttatgcagaccagcaagttgcaaaacggattttccaaaagctgatctcctatgttgatgacggtaaaggaagcttcaagcagcctgacagcgtcgtgaaagaaacagttgagaaaggctcgaaccccgccgctttagccggaccgaatacgccggatgataaaaaagtgacggagtacttcgtgaaaggaacggagcctactgccgtttctaaaacgtacaagaaagaaaaagccaataaaccgacaggcttgcaagtgaagtacgacaaagacaaaaaatcattaaacttaagctgggattatgatggtgatgcaacattcaatgtcaaacaatctgttgatggcggaagctacagcgacattcaaaacagttcggcaaaagaagccgtcgtctcagatgtcacccctgggtccacatacagcttccaggtgacagcggaaaatgatgacggaaagagcgatacggcatcagcatctttcaaagttccgaaagatgaagaagacaatgcagataaagataaagcgactgacgatgaacagaaacctgatgataaggataaagacaagacccaaaccgatgattcaaaaacggacgacaatcagacggatacgtcagacggatcggatcaaacacctactgatgacccgaataaaaatccggatcaatccaatcaggatcaaacggataataacaatccgaatcagcctggtaacgggacggataacaacggacagcaagataacagctctgacaatagttccgataccggttcagatggttcaaaaaatcctgacagcggccagaaggataaaaaccaagactcatcttcaactgtcaacggccagtcaaattcgtcaaccatccagtcaaataccgctaattag

SEQ ID NO: 14 - DNA, 2847 bp, synthetic construct
  source 1..2847: mol_type = other DNA
atgtcagataattatcgctctcgtgaagaacgacgaagagctatgaatgacaacaaaccagaaggtaaatctcaaggaaaaccgaaaaagaagaaaaaaggaggcctatttcgtaaaattgttgccgcagttttactaataggaatcatcgggctcattgccggagtgggaactttttttgctatgataagcgatgctccaaaagttgatgactcggtattaaaaaactctttttcatctaaagtatatgcaaacgatggaaagacagttgtaaaagaaattggagctcaaaaacgtacatatgtgcaatacgatgaaatccctcaagtcgtcaaaaacgcatttatcgctacggaagacgttcgtttttataaacatcacggcgtcgacttttaccgaattggcggagcattaatggcgaactttaaaaacggcttcggtgctgagggcggtagtacgattactcagcaagtggtaaagaattcgttcttatcaccaaagaagacggttaaacgtaaagtacaagaaatgtggttagcttaccagcttgagcgtaaatattctaagcaacaaattcttgaaatgtatttaaacaaaatatatttcgcatcaggtgaagtatacggtattgaacgagcagctgagaaattttacggagttaaaagcgtaaaagatttaactcttgatgaagctgctatgttggcaggcttaccaaaagcgccaactacttataacccagttacaaacccagagaatgctactaaacgccgtaacacagtattaaacttaatggctaaaaacggcttcattacacaagccgaggccgacaaagcaaaacaagtagatgtacaggctcacttagctaaacaaactgaatcaacatctaagtacgatgtatttattgatcaagtaatcgaggaagtaaaaaagaaaacagacgcggatccattttctgcgggcttagaaatttatacaacgcttgacactaatgctcaggactatgtggatgatgtgatgaataacaaagtagtcaacttcccgaacgataaattccaagcaggtcttgtcgttgtagataatgaaacaggcggtattcaagcaattggaggcggacgtaatcgtgtatcaggcggcttaaactttgcaacaaatatgagacgccagcccggctcaacaattaagccgatcttagattacggtccagcgattgagaacttaaagtggtccactggacaaccgctaaaagatgaagaatatcattattcaaacggcacgccaatccgaaacttcggacgcaattataaaggctgggtttcagcgcgtgaagctttaggacgctcactgaacatccctgctttaaaagcgtttcaagcaaccggtgcggacaaagcaaaagagttcgctacgggccttgggctaaaattagacgaagaagacggcgaagcttattctgaatcttattctatcggaggattccgtacaggtgttacacctctacaaatggctggagcctatagcgcttttgcaaatgaaggtatgtacaatgcaccgcatacagtgacaaaggtaaaattccctgatggaacagaaattgatttaacaccaaaatcaaaacgcgcaatgcaagattacacggcatttatgatgacggacatgatgaaatctgttgtaaacgaatcatatggtacagctcgtgctgttcgtacaccaggattagaaattgcgggtaaaacgggaacttcgaactttagtgcggcagaaaaacaacgatacaacattccagacgctggtgaaaaagacgtttggtttaatgggtttacgccaaactatacgatttccgtatggacaggctatgaaactgctaaagacggctatattttaagtcctaatgaaacaggcatggcaaaaaatatctttaagcatgttattactcatatttctgaaggcaaacctaaaggaagtttcaagcagccggacagcgtagttgaacgttccgtggaaaaaccaacgggcaaagacttaaaacgcccaagcggctctactccttcgagcttaattactaaagaatactttgtacgcggtactgcaccgacaaaagtatctcaaacgtttgtgaagaaagaaaaagaagaaaagaaagaagagaaagaagagaaaaaagaggacaaaaaagagaaaaaagacaataaaaatgtgaagccaaaaattactgcttcgtatagcggaggagcaatttcagttaactggtcagcgtcaaacgcagccggtgatacgacctttacagtcacaatgtcatctcctaacggttctgaacaactcgcatctggcgcaggagccggtggaaaaacaatttccaacccagcaccaggcgcaacgtatagtttcaccgtaacaacaaatacgggcgaaagcgctagttcttctgtaaaagtccctgattcatcagatcaagctaccgaagatgattctactaagcctgaagatgatggaacaacggacgataatcaaaaggaagacaatagcaacactgatcaaaacaacggctctgatcaaggtgacgattcatctactcagccggatgataaccaaaacagcaataatggcagcaataatggcagcaataacggcggtaacaatagcggcaataccgataataacaataatgatagcggcaatgctaacgatggtaacagcaacaacaacaataataacggaggcacaacaactcctccagcagacaacggtggaagtgactcaggaagtcagacaccaccaacaacccctgctacaccatcgacgccgtcaacacctgctacaccgcagcaagagccaacaactgaaaattaa

SEQ ID NO: 15 - DNA, 2676 bp, synthetic construct
  source 1..2676: mol_type = other DNA
atgtctggtgaatatcgttctcgtgtggaacgaaagcaggcagcgaaacagacgaaacaaaagaaggcgaaaaagaaaagaggaaggtcctggttgaaagcgatcgccattgcgatagtgctgatgatcgcccttggcgccatcgggggggtggctgcgttcgcctactttgtcaaggacgcccctccgctcgatgaggcgaaaattaaagatccgctctcgtcgaccgtgtacgatatgagcggaaagaaagtcgccgagctgggcggttacaaacggacgtacatctcatacaaggacgtgccgaaagtgctggaaaatgcggtattggcgaccgaagacgcccgcttttacgagcatcacggcatcgacatcgtccgtctagccggcgcggtcatcgccaatattcgcgaagggttcggcgcggaaggcggcagcacgatcacccagcaagtcgtgaaaatgacgttcctctcaccaaagaaaacgttggagcggaaagcgcaagaagcgtggctcgccttgcagcttgagcaaaagtattcgaagcacgaaattttggaaatgtacttgaacaaaatttattactcagacggcatttatggcgtcgcccgggcggcggaatattacttcggcaaaacgaacttaaaagagctgacgctccccgaggcagcgctcttggccggcatgccgcaaagcccgaaccggtacaacccgtacgttcatccagaggcggcgaaaaaacggcgcgacactgtcttgtcgctcatggtgaaacatggctttatcagcgcggaagaagcggaaaaagcgaagcaggtgccgatcaaatcgatgctcaccgagcggaaaaagccgcaaaattccgtgccatacgacgccttcattgacgaagtgatcaaagaagtgacggacaaagccaatgtcaacgtctttgaagacgggctgaaaatttacacgacgcttgatcaagaggcgcaatcgtacgttgaaaagctattaaactccgatcaacgttttaccgccaaaaaagatttgcagtcgggaatcgccctcgtcgacacgaaaacgggcgccattcgcgcgttaggcgggggacgccatcgcgacaacatcgagttcggtttcaactacgcaattcagcctgtcggccagcccggttcaacgattaagccgattttggactacgggccggcgatcgaatatttaaaatggtcaaccgcgcacattcttgttgacgagccgtatacgtactccgacggcaccccgatccgcaacgccggcgggcgctacgccggaccggtgacggtgcgcgatgccctcacatggtcgcgcaacatcccggcgttaaaaacgttccaagcggtcggcaaggagcgggcgagagagtttgccaaccggctcggcatgggctttgacgaagtatatgagccgtacgcaatcggcggcgtatcgcgctacgtatcgccgctgcagatggccggcgcctacgccgccttcggcaacaacggcgtttacacgaagccgtatgcggtgacgaaaatcgtcttcccagacggcacggaaatggacttaaagccaaaaccgaagcgagtcatgcatgattacaccgcatacatgattaccgacatgctcaaatcggtcatccgctctggcactgggcagtccgcctatgtcccagggttggagctcgccggcaagaccggaacgacaaactacggcaaagacggcgcgaagtttggcctcaaacgggacgatgtgccggacagctggattgtcggctatacgccaaactacacggcggccatttgggtcggcttcagcaaaaaaagcgaaacagctacattaggggatcgggagcagcgcattccgaagctcttgctgaaagatctgatgtcccatctcgacgatggcagcggagagtttcagcggccggacagcgtcgtcaaactgccgatcaaaaaaggcacgaatccgccgaaacttgccggcaaatacacgcctgaaagcgacattacgtatgaattgttcgtgcgcggcaccgaaccgacagaagtcgcaaaagatgagccggaagagctcccggctgtcaaagggttggccgcttcgtacgatcaagctgccaacatcatttccgtctcttggtcgtatgatgagaaaaccgacgacacgctgtttgaagtgcgcatcaaagacgagcaagggcatacggaaacggtcacgaccaaagatttaggcgtgacagtgacgaacgtggcgccaggggcttcgtacacaatcgccgtctatgccaaggaaggcgagcggatgagcaagccggcgatcacgagcatccgcatccctggcggcgagcagccgacggcgccgggcaatgaagcgaacgatgatggcaacaaccacggcgccggaaatggcggcacgaacggggataacggcgatgatcatgggggcaatggaaacaatcaaaataacaacaatggcggaaatagcggaaacaacggcaatggcaacgaagatcgcgatgatggcggcgaagaggacaacggcggcgaagacaacggccaaggggatggcggtgcaacaacgccgacgacaccaacaaccccaacgacgccgaccaatccacaatcaggaaacagcggcaatgctggcatgagaaatggaagccgccagtga

SEQ ID NO: 16 - DNA, 2163 bp, synthetic construct
  source 1..2163: mol_type = other DNA
atgtcagatcaatttaacagccgtgaagctcgacgaaaagcgaacagcaaatcgagtccttcaccgaaaaaaggcaagaaacgaaaaaagggcggattgtttaaaaagacccttttcactttactcattctgtttgttttaggcgtagtcggcggtgctgttacatttgccgtcatggtttctgatgcgccgtcccttgatgaaagcaaattgaagacgccttattcatcaacgatttatgataaaaatggaaaagagatcgccgaagtcggcgccgaaaaacggacctacgtctcgatagatgaaattcccgatgttgtaaaagaagcctttatcgcgacagaagacgctcgtttttatgagcaccacgggattgaccctgtccgtatcggcggtgcattagttgccaactttaaagacggcttcggggctgaaggcggaagtacgatcacccagcaggtcgtcaaaaactcccttctttcacatcagaagacgttgaaacggaaggtgcaggaagtatggctttcgattcagctggagcgcaattactctaaagatgaaattttagaaatgtatttaaaccggatttatttttctcctagagcatacggaatcggaaaagcggcggaagaatttttcggcgttactgatttaagcaaattgactgtcgaacaggctgcgacgcttgcaggaatgccgcagagcccaacagcgtacaacccggtcaaaaacccggataaagcggaaaaacgacggaacatcgtactcagtctgatgaaaaagcaaggatttatttctgattctcaatataacaaagcgaaaaaagtggcagtgaaagacgaaggcgtcgtgtcacagaaggaatatgaaaaagcaagtacaaacaaatacagcgcatttgttgaagaggttatgaaggaaattgatgaaaaatctgatgtcgatccatctgctgacggattaaagatctatacgacattagatacaaaagcacaagataaactagatgaattaatggacggagacaccgtcggatttactgaaggcatgcagggcggtgtgacgcttctcgataccaaaaacggagaagtccgagcgattggtgcgggacgcaatcagcctgtcggaggctttaactatgctactcaaactaaggcacagcctggttcgaccataaaaccgattttggactacggaccagttattgaaaataagaaatggtccacatacgaacaaattgatgactcagcttatacgtattctaacggaaaaccgattcgtgattgggaccgcaaatatctagggcctatctcaatgcgttacgctctggcccaatcaagaaatatacctgctttaaaagcattccaggcagtcggtaaagatactgctgtagactttgcaaatggactcgggcttggtttaacaaaagataatgtaacagaggcctattctattggcggtttcggtgggaacgatggtgtttctcctctgacaatggcaggtgcatacagcgcgtttggaaataacggaacgtataatgaaccgcattttgtaaaatctatcgaatttaacgatggcacgaagcttgacttaacaccaaaatcaaaatcagccatgagtgattatactgcgtttatgattacagatatgctgaaaacagctgtgaagacaggtactggacagcttgcacaagtacctggtgtagaagttgcaggaaaaacaggaacgactaactttgatgataatgaagtcaaaaggtacaatatcgctagcggtggcgcccgagattcttggttcgttggctacacaccgcaatatacagctgccgtctggacgggaatgggagaaaacgaggctggaaagaaatcactttcagctgaagagcaaaaagttgcaaagcgcatcttcgcccagctcatcgccgatgtcgatgacggaagcggatcatttgagaagcctgacagcgtagtggaagccaccgtagaaaaaggttctaatccggcaaaactggcagggccaaatacgccaagcgataagaagctcacagagtactttgttaaaggcacagctccttctactgtttctaaaacatatgagaaagaagaaaaagaggaaacagctaaactgtctggtttaaacgtgaaatacgataaagacaatcaa

SEQ ID NO: 17 - DNA, 2100 bp, synthetic construct
  source 1..2100: mol_type = other DNA
atgtcagatcaatttaacagccgccaggaacggcgaaaagcccagcagggcaaaagcaggtctaatactcattctaaacccaagaaaaagaaaaaagcaggattgtttaagaaaattcttttatccattttaatcatcggtgttatcggcttgattgccgggggcgtcacttttgcggtgatggtagcggactcgccgtcccttgatgaagcaaaactgaagacaccgtactcttctaccatctatgacaaaaacggaaaagaaatcgccgaaatcggttcggaaaaacggacgtacgtgtcgattaaagacatcccggattccgtcaaaaacgcatttcttgcaacggaagacgcccgtttctatgaccaccacggcgtcgacccgatccggatcggaggagcgcttctggcaaacttcgaaggcggatttggttctgaaggcggaagtacgatcacacagcaggtcgtcaaaaactcccttctgtcacatgaaaaaacgctgaaacggaaagtccaggaagtctggctgtctttccagctggaaagaaaatactcaaaagacgaaattcttgaaatgtatttaaatcggatttacttctcgccgcaggcctacggtgtcggaaaagcggctgagcagttctacggggttacagatctaaacgacttaacggtcgagcaggccgcaacgcttgccggcatgccgcaaagcccgaataactacaacccgatcaaacatccggaaaaagccgagaaacggcgcaatgtcgtgctgagcctgatgaataaacacggctttatctctgacgcagagtacaacaaagcgaaaaaagtagccgtcacaaaaggccttgtatctccgaaagagtacgcaaaaacaacgtccaacaaatacagcgcgtttatcgagcaggccgtcgccgaagtgaaagaaaaagcgaacgtcactccgggaacggacggactgaagatttacacgacgattgatacagacgcacaggattatgtggatgagctgatggacggcgattctattcagtataccgaaaaaatgcaggccggcctcaccctgctcgatacgaaaaccggagagatccgcgcgctcggcggcggacgcgaccggaaagcaggcgactttaactatgcagtagatactaaacgtcaacccggttcaacgattaagccaatccttgactacggccctgtcattgagaacaagaaatggtcgacgtatgaacagattaaagatgaagcatacacgtattcaactggagatccgattaacaactatgacagaagctacagaggctggatatcgatgagagaggcgctggtcgattctcgaaacatcccggctttaaaagcgttccaggcagccggcaaagacaatattgtaaaatttgcaggcaaccttggccttaacataaattcagatgacttagtggaagcttatgcaatcggcggcttcggaaatggtgtgtcgcctctgcaaatggccggagcgtacagcgccttcggcaacaacggctactacaatgagccgcatactgttaccgcggtggaattcaatgacggcactaagcttgatttgacgcctgaatcaaaggctgccatgagcgattataccgcatttatgataacggatatgctgaaaagcgctgttcagcgaggaacaggaacagcggctcaagtccccggcgtcactgtagcaggtaaaacgggtacgacgaactttaccgaggacgacattagaaaacacggcataaatcgaaacggagcgagagattcatggttcgtcggctatacgccgcaatataccgcagcggtctggaccggtaaagaaggctttaattcgctgagccagagcgaacagcaagtcgccaagctcctattcaaaaaactgattgccaaggtggacaacggcagcggctcatttgaaaagcctgacagcgtcgttgaagcgaccgttctaaaaggctcgaacccgccggtgctcgccagcagcaacacgcctagcgataaaaaagtcactgagtactttgtaaaaggcacacagccgacgaccgtttcgaaaaaatatgaagaaaaagaaaatgcctga

SEQ ID NO: 18 - DNA, 2133 bp, synthetic construct
  source 1..2133: mol_type = other DNA
atgtcagatcaatttaagagccgtgaagaacgccgaaaggcaaggcagacgaaaagccccaagccttcttcaccgaaaaaaggcaagaaacagcgtaaaggcggtttattcaaaaagattgtactgtccttggttattctgtttgtcctcggcatagtcggcggagctgcggcatttgccgttttagtttccggtgcgccgtctttggacgaagccaagctgaaaacgccttactcttcaacaatttacgataaaaacggcaaagaaatcgcggaagtgggcggcgaaaaacgaacctacgtcccgattaaggatatacccgatgttgtgaaggaagcttttatcgcaacagaggacgcccgtttttataagcaccacggaatagacccgatccggatcggaggcgccttggtcgccaactttacggatggtttcggttcagaaggcggaagcacaatcacccagcaggttgtgaaaaactcgcttctgacccaccagaaaacgattaaacggaaagctcaggaagtatggctttcactgcagctggagcgccattattctaaagatgaaattttagaaatgtatttaaacaaaatttatttctcaccgcgcgcctacggcgtcggtaaagccgctgaagactttttcggcgtgaaagatttgagcaagcttacggttgaacaggcagcaacccttgccggaatgccgcaaagtccgaccggatacaacccggttaaaaatccgaaggcttctgaaaaacgccggaacatcgttctcagcttaatggaaaaacaaggatttatctcaaattctgaataccaaaaagcaaagaaagtgtccatgaaagatgaaggcgtcgtcagccaaaaggaatacgataaaaaggacacaaataaatacagcgcttttgtcgaagaagttatgaaagaaattgagacaaaagaaaagaaagacgtcgatatttcaactgacggattaaaaatttacacgactcttgatacgaaagcacaagattatcttgatcaattgatgaacggcgacacagtcggctttacccaaggcatgcagggcggcgtcaccctccttgatacgacaaacggagaaatccgcgccatcggagcaggacgaaacagaactgccgggggatttaactacgcgacagacactcaacgccagccaggttcaacgataaaaccgattcttgattacggtccggtcattgaaaataaaaaatggtcgacgtatgagcagattgatgactcagcctatacgtactctgacggcactccgattcatgactatgacaataaatacagacaaatgataacaatgcgagaggctctctcagactcacgtaatattcctgctctgaaggcattccaagcagcaggtaaagataatgccgtcgacttcgcaaacgacttaggactaggattaaacaaagacagtgtaacagaatcttattcgatcggcggttttggagctgataacccgggcgtttccccgctgactatggctggtgcctacagcgcttttggaaataacggaacgtacaatgaaccgcacgcggtcacatcgattgaatttaatgacggaactaagcttgatttaacaccgaaaccgaagagtgctatgagcgattacacggcattcatgattaccgatatgctaaaaacagtggtgcagtcaggaacgggtaaactggctcaagtaccgaacgtggaagtcgccggtaaaacgggtacgacaaactttactaaagaggatattcaaaaatataacattgctccaggcggagtaccggattcatggtttgtcggctacacccctcaatatacagcggcggtttggaccggtgttgagtctggtaacaaagcaggtaaaaaatcattatcttatgcagaccagcaagttgcaaaacggattttccaaaagctgatctcctatgttgatgacggtaaaggaagcttcaagcagcctgacagcgtcgtgaaagaaacagttgagaaaggctcgaaccccgccgctttagccggaccgaatacgccggatgataaaaaagtgacggagtacttcgtgaaaggaacggagcctactgccgtttctaaaacgtacaagaaagaaaaagcctag

SEQ ID NO: 19 - DNA, 2115 bp, synthetic construct
  source 1..2115: mol_type = other DNA
atgtcagataattatcgctctcgtgaagaacgacgaagagctatgaatgacaacaaaccagaaggtaaatctcaaggaaaaccgaaaaagaagaaaaaaggaggcctatttcgtaaaattgttgccgcagttttactaataggaatcatcgggctcattgccggagtgggaactttttttgctatgataagcgatgctccaaaagttgatgactcggtattaaaaaactctttttcatctaaagtatatgcaaacgatggaaagacagttgtaaaagaaattggagctcaaaaacgtacatatgtgcaatacgatgaaatccctcaagtcgtcaaaaacgcatttatcgctacggaagacgttcgtttttataaacatcacggcgtcgacttttaccgaattggcggagcattaatggcgaactttaaaaacggcttcggtgctgagggcggtagtacgattactcagcaagtggtaaagaattcgttcttatcaccaaagaagacggttaaacgtaaagtacaagaaatgtggttagcttaccagcttgagcgtaaatattctaagcaacaaattcttgaaatgtatttaaacaaaatatatttcgcatcaggtgaagtatacggtattgaacgagcagctgagaaattttacggagttaaaagcgtaaaagatttaactcttgatgaagctgctatgttggcaggcttaccaaaagcgccaactacttataacccagttacaaacccagagaatgctactaaacgccgtaacacagtattaaacttaatggctaaaaacggcttcattacacaagccgaggccgacaaagcaaaacaagtagatgtacaggctcacttagctaaacaaactgaatcaacatctaagtacgatgtatttattgatcaagtaatcgaggaagtaaaaaagaaaacagacgcggatccattttctgcgggcttagaaatttatacaacgcttgacactaatgctcaggactatgtggatgatgtgatgaataacaaagtagtcaacttcccgaacgataaattccaagcaggtcttgtcgttgtagataatgaaacaggcggtattcaagcaattggaggcggacgtaatcgtgtatcaggcggcttaaactttgcaacaaatatgagacgccagcccggctcaacaattaagccgatcttagattacggtccagcgattgagaacttaaagtggtccactggacaaccgctaaaagatgaagaatatcattattcaaacggcacgccaatccgaaacttcggacgcaattataaaggctgggtttcagcgcgtgaagctttaggacgctcactgaacatccctgctttaaaagcgtttcaagcaaccggtgcggacaaagcaaaagagttcgctacgggccttgggctaaaattagacgaagaagacggcgaagcttattctgaatcttattctatcggaggattccgtacaggtgttacacctctacaaatggctggagcctatagcgcttttgcaaatgaaggtatgtacaatgcaccgcatacagtgacaaaggtaaaattccctgatggaacagaaattgatttaacaccaaaatcaaaacgcgcaatgcaagattacacggcatttatgatgacggacatgatgaaatctgttgtaaacgaatcatatggtacagctcgtgctgttcgtacaccaggattagaaattgcgggtaaaacgggaacttcgaactttagtgcggcagaaaaacaacgatacaacattccagacgctggtgaaaaagacgtttggtttaatgggtttacgccaaactatacgatttccgtatggacaggctatgaaactgctaaagacggctatattttaagtcctaatgaaacaggcatggcaaaaaatatctttaagcatgttattactcatatttctgaaggcaaacctaaaggaagtttcaagcagccggacagcgtagttgaacgttccgtggaaaaaccaacgggcaaagacttaaaacgcccaagcggctctactccttcgagcttaattactaaagaatactttgtacgcggtactgcaccgacaaaagtatctcaaacgttttaa

SEQ ID NO: 20 - DNA, 2085 bp, synthetic construct
  source 1..2085: mol_type = other DNA
atgtctggtgaatatcgttctcgtgtggaacgaaagcaggcagcgaaacagacgaaacaaaagaaggcgaaaaagaaaagaggaaggtcctggttgaaagcgatcgccattgcgatagtgctgatgatcgcccttggcgccatcgggggggtggctgcgttcgcctactttgtcaaggacgcccctccgctcgatgaggcgaaaattaaagatccgctctcgtcgaccgtgtacgatatgagcggaaagaaagtcgccgagctgggcggttacaaacggacgtacatctcatacaaggacgtgccgaaagtgctggaaaatgcggtattggcgaccgaagacgcccgcttttacgagcatcacggcatcgacatcgtccgtctagccggcgcggtcatcgccaatattcgcgaagggttcggcgcggaaggcggcagcacgatcacccagcaagtcgtgaaaatgacgttcctctcaccaaagaaaacgttggagcggaaagcgcaagaagcgtggctcgccttgcagcttgagcaaaagtattcgaagcacgaaattttggaaatgtacttgaacaaaatttattactcagacggcatttatggcgtcgcccgggcggcggaatattacttcggcaaaacgaacttaaaagagctgacgctccccgaggcagcgctcttggccggcatgccgcaaagcccgaaccggtacaacccgtacgttcatccagaggcggcgaaaaaacggcgcgacactgtcttgtcgctcatggtgaaacatggctttatcagcgcggaagaagcggaaaaagcgaagcaggtgccgatcaaatcgatgctcaccgagcggaaaaagccgcaaaattccgtgccatacgacgccttcattgacgaagtgatcaaagaagtgacggacaaagccaatgtcaacgtctttgaagacgggctgaaaatttacacgacgcttgatcaagaggcgcaatcgtacgttgaaaagctattaaactccgatcaacgttttaccgccaaaaaagatttgcagtcgggaatcgccctcgtcgacacgaaaacgggcgccattcgcgcgttaggcgggggacgccatcgcgacaacatcgagttcggtttcaactacgcaattcagcctgtcggccagcccggttcaacgattaagccgattttggactacgggccggcgatcgaatatttaaaatggtcaaccgcgcacattcttgttgacgagccgtatacgtactccgacggcaccccgatccgcaacgccggcgggcgctacgccggaccggtgacggtgcgcgatgccctcacatggtcgcgcaacatcccggcgttaaaaacgttccaagcggtcggcaaggagcgggcgagagagtttgccaaccggctcggcatgggctttgacgaagtatatgagccgtacgcaatcggcggcgtatcgcgctacgtatcgccgctgcagatggccggcgcctacgccgccttcggcaacaacggcgtttacacgaagccgtatgcggtgacgaaaatcgtcttcccagacggcacggaaatggacttaaagccaaaaccgaagcgagtcatgcatgattacaccgcatacatgattaccgacatgctcaaatcggtcatccgctctggcactgggcagtccgcctatgtcccagggttggagctcgccggcaagaccggaacgacaaactacggcaaagacggcgcgaagtttggcctcaaacgggacgatgtgccggacagctggattgtcggctatacgccaaactacacggcggccatttgggtcggcttcagcaaaaaaagcgaaacagctacattaggggatcgggagcagcgcattccgaagctcttgctgaaagatctgatgtcccatctcgacgatggcagcggagagtttcagcggccggacagcgtcgtcaaactgccgatcaaaaaaggcacgaatccgccgaaacttgccggcaaatacacgcctgaaagcgacattacgtatgaattgttcgtgcgcggcaccgaaccgacagaagtcgcaaaagatgagccggaagagctctga

SEQ ID NO: 21 - protein, 545 aa, Bacillus subtilis
MKKRWSIVTLMLIFTLVLSACGFGGSGSNGEGKKDSKGKTTLNINIKTEPFSLHPGLANDSVSGGVIRQTFEGLTRINADGEPEEGMASKIETSKDGKTYTFTIRDGVKWSNGDPVTAQDFEYAWKWALDPNNESQYAYQLYYIKGAEAANTGKGSLDDVAVKAVNDKTLKVELNNPTPYFTELTAFYTYMPINKKIAEKNKKWNTNAGDDYVSNGPFKMTAWKHSGSITLEKNDQYWDKDKVKLKKIDMVMINNNNTELKKFQAGELDWAGMPLGQLPTESLPTLKKDGSLHVEPIAGVYWYKFNTEAKPLDNVNIRKALTYSLDRQSIVKNVTQGEQIPAMAAVPPTMKGFEDNKEGYFKDNDVKTAKEYLEKGLKEMGLSKASDLPKIKLSYNTDDAHAKIAQAVQEMWKKNLGVDVELDNSEWNVYIDKLHSQDYQIGRMGWLGDFNDPINFLELFRDKNGGNNDTGWENPEFKKLLNQSQTETDKTKRAELLKKAEGIFIDEMPVAPIYFYTDTWVQDENLKGVIMPGTGEVYFRNAYFK

SEQ ID NO: 22 - protein, 335 aa, Bacillus subtilis
MNAKRWIALVIALGIFGVSIIVSISMSFFESVKGAQTDLTSLTDESQEKTLENGSPSSKIAVLEVSGTIQDNGDSSSLLGADGYNHRTFLKNLERAKDDKTVKGIVLKVNSPGGGVYESAEIHKKLEEIKKETKKPIYVSMGSMAASGGYYISTAADKIFATPETLTGSLGVIMESVNYSKLADKLGISFETIKSGAHKDIMSPSREMTKEEKNIMQSMVDNSYEGFVDVISKGRGMPKAEVKKIADGRVYDGRQAKKLNLVDELGFYDDTITAMKKDHKDLKNASVISYEESFGLGSLFSMGANKMFKSEIDFLNMREILSQSGSPRMMYLYAK

SEQ ID NO: 23 - protein, 450 aa, synthetic construct
MKLAITAKAAAGAHMAKYLELEEGGVIMQAFYWDVPGGGIWWDHIRSKIPEWYEAGISAIWLPPPSKGMSGGYSMGYDPYDYFDLGEYYQKGTVETRFGSKEELVRLIQTAHAYGIKVIADVVINHRAGGDLEWNPFVGDYTWTDFSKVASGKYTANYLDFHPNELHCCDEGTFGGFPDICHHKEWDQYWLWKSNESYAAYLRSIGFDGWRFDYVKGYGAWVVRDWLNWWGGWAVGEYWDTNVDALLSWAYESGAKVFDFPLYYKMDEAFDNNNIPALVYALQNGQTVVSRDPFKAVTFVANHDTDIIWNKYPAYAFILTYEGQPVIFYRDFEEWLNKDKLINLIWIHDHLAGGSTTIVYYDNDELIFVRNGDSRRPGLITYINLSPNWVGRWVYVPKFAGACIHEYTGNLGGWVDKRVDSSGWVYLEAPPHDPANGYYGYSVWSYCGVG

SEQ ID NO: 24 - protein, 516 aa, synthetic construct
MRKKITLACKTCGNRNYTTMKSSASAAAGAHMELMAAPFNGTMMQYFEWYLPDDGTLWTKVANEANNLSSLGITALWLPPAYKGTSRSDVGYGVYDLYDLGEFNQKGTVRTKYGTEAQYLQAIQAAHAAGMQVYADVVFDHKGGADGTEWVDAVEVNPSDRNQEISGTYQIQAWTKFDFPGRGNTYSSFKWRWYHFDGVDWDESRKLSRIYKFRGKAWDWEVDTEFGNYDYLMYADLDMDHPEVVTELKNWGKWYVNTTNIDGFRLDAVKHIKFSFFPDWLSYVRSQTGKPLFTVGEYWSYDINKLHNYITKTDGTMSLFDAPLHNKFYTASKSGGAFDMRTLMTNTLMKDQPTLAVTFVDNHDTEPVQALQSWVDPWFKPLAYAFILTRQEGYPCVFYGDYYGIPQYNIPSLKSKIDPLLIARRDYAYGTQHDYLDHRDIIGWTREGGTEKPGSGLAALITDGPGGSKWMYVGKQHAGKVFYDLTGNRSDTVTINSDGWGEFKVNGGSVSVWVPR

SEQ ID NO: 25 - protein, 613 aa, synthetic construct
MFAKRFKTSLLPLFAGFLLLFHLVLAGPAAASAEAMASPLTKPSTPIAATNIQKSADFPIWWKQAVFYQIYPRSFKDSNGDGIGDIPGIIEKLDYLKMLGVDAIWINPHYESPNTDNGYDISDYRKIMKEYGSMADFDRLVAEMNKRGMRLMIDIVINHTSDRHRWFVQSRSGKDNPYRDYYFWRDGKQGQAPNNYPSFFGGSAWQLDKQTDQYYLHYFAPQQPDLNWDNPKVRAELYDILRFWLDKGVSGLRFDTVATFSKIPGFPDLSKAQLKNFAEAYTEGPNIHKYIHEMNRQVLSKYNVATAGEIFGVPVSAMPDYFDRRREELNIAFTFDLIRLDRYPDQRWRRKPWTLSQFRQVISQTDRAAGEFGWNAFFLDNHDNPRQVSHFGDDSPQWRERSAKALATLLLTQRATPFIFQGAELGMTNYPFKNIEEFDDIEVKGFWNDYVASGKVNAAEFLQEVRMTSRDNSRTPMQWNDSVNAGFTQGKPWFHLNPNYKQINAAREVNKPDSVFSYYRQLINLRHQIPALTSGEYRDLDPQNNQVYAYTRILDNEKYLVVVNFKPEQLHYALPDNLTIASSLLENVHQPSLQENASTLTLAPWQAGIYKLN

SEQ ID NO: 26 - DNA, 1638 bp, synthetic construct
  source 1..1638: mol_type = other DNA
atgaaaaaacgttggtcgattgtcacgttgatgctcattttcactctcgtgctgagcgcgtgcggctttggcggcagcggatcaaacggtgaagggaaaaaggacagtaaaggaaagacgacacttaacattaatattaaaactgagccgttctccttacatccgggattggcaaatgattcagtatcaggcggtgttatccgtcagacttttgaaggattgacacgtatcaatgcagatggtgagcctgaagaaggcatggcttctaaaattgaaacgagcaaggacggaaagacatatacatttaccattcgtgatggtgtgaaatggtctaatggagaccctgtaactgcacaagattttgaatatgcttggaaatgggcgcttgaccctaataatgaatcacaatacgcttaccagctctactacataaaaggtgctgaagcggcgaataccggaaaaggcagcctagacgatgtggcagtaaaagctgtgaatgacaaaacgctgaaggttgaattaaataacccgactccatatttcactgaattaactgcgttctatacgtatatgccgatcaataagaaaattgcagagaaaaataaaaagtggaatacaaatgccggagatgattatgtatcaaacgggccgttcaaaatgacggcatggaaacacagcggctctattactctcgaaaaaaatgaccagtattgggataaagacaaagtcaaactgaagaaaatcgatatggttatgatcaacaataacaatacggaactaaaaaaattccaagctggcgaacttgattgggccggtatgccgctcggacagcttccgacagaatccctgccgaccctgaaaaaagacggttctttacatgttgagccgattgcaggagtgtattggtacaaattcaacactgaagctaagccattagacaacgtcaatatccgtaaagctttaacatattcgcttgaccgtcagtcgattgttaaaaacgttacgcaaggagagcaaatcccggcaatggctgcagtgccgcctacaatgaagggatttgaggataacaaagaaggatacttcaaagacaatgatgtcaaaacagcaaaagaataccttgaaaaaggcctaaaagaaatgggcttaagcaaggcatctgatttgccaaaaatcaaattgtcttacaacactgatgacgcacacgcgaaaatcgctcaagcagtacaagaaatgtggaagaaaaatttaggcgttgatgttgagcttgataactcagagtggaatgtctatattgataagctccacagccaagattatcaaatcggccgtatgggctggctcggcgacttcaatgatcctatcaacttccttgaattgttccgcgacaaaaacggaggaaataacgatacaggctgggaaaatccagaattcaaaaagcttctgaatcagtcacaaactgaaacagataaaacaaaacgtgcagagctgctgaaaaaagcagaaggtattttcattgatgaaatgccggttgccccaatctatttctatactgatacttgggtacaggatgaaaacctaaaaggtgttatcatgccaggtactggtgaggtttatttcagaaacgcatattttaaataa

SEQ ID NO: 27 - DNA, 1008 bp, synthetic construct
  source 1..1008: mol_type = other DNA
atgaatgcaaaaagatggattgcattagtgattgctctggggattttcggcgtgtctattatcgtcagcatctctatgagtttctttgaaagcgtcaaaggcgctcaaacggatctcacatcactgacggatgaatcgcaggagaagacgctggaaaacggcagtccctcaagtaaaattgccgtattagaggtcagcggcaccattcaggataatggggactcaagcagtctgcttggtgcagacggatataaccacagaacgttcttaaaaaaccttgagcgcgcaaaagatgacaagacggtcaaaggtatcgttctgaaggtgaattctccgggtggcggagtgtatgaaagtgctgaaatacataagaaactggaagaaatcaagaaagaaacgaaaaaaccgatttacgtgtcaatgggttcgatggcagcatcaggaggctattacatctcaacagcggctgataagatttttgcgacaccggaaaccctgaccgggtcactcggcgtcattatggaaagcgtcaattattcaaagcttgccgacaagcttggcatttcttttgaaacgattaagagcggggcccataaggacattatgtctccttcccgtgagatgacgaaagaagaaaaaaacatcatgcaatcgatggttgataattcgtatgaaggctttgttgatgtcatttcaaaagggcgcggcatgccgaaagcagaggtaaagaaaattgcggacggccgcgtgtatgacggacggcaggcgaaaaaactgaacctcgttgatgagcttggtttttatgacgataccattacggccatgaaaaaggatcacaaggatttgaaaaacgcctctgtcatttcttatgaggaaagcttcggattaggctcactgttttccatgggcgcgaacaaaatgtttaaaagtgaaattgattttttgaatatgagagaaattctatcgcaatccggttcgccgagaatgatgtatctctatgcgaagtag

SEQ ID NO: 28 - DNA, 1353 bp, synthetic construct
  source 1..1353: mol_type = other DNA
atgaaactggcaatcacagcgaaagcggcggccggtgcacatatggccaaatacttagaactcgaagagggcggtgttataatgcaagcattctattgggatgttccaggaggcggaatctggtgggaccacatcagaagcaagattcctgaatggtacgaggccggcataagcgccatctggctgccacctccttctaaaggaatgagtggaggatatagtatggggtacgacccatacgactactttgacctgggtgaatattatcagaaaggcacagtagagacacgctttggatcaaaggaggaattagttcggttgattcaaacagctcatgcttatgggattaaggttatcgccgatgtggttatcaaccatagagctggaggcgatcttgaatggaacccattcgttggtgattatacatggacagatttttctaaggttgcttctggaaagtatactgcgaactacttggacttccacccaaatgagcttcactgttgtgacgagggtacctttggtgggttcccggatatatgtcaccacaaggagtgggatcaatactggctttggaaatcaaacgaatcttacgcggcatatttacggagcatagggttcgacgggtggcgtttcgactacgtgaagggatacggtgcttgggtggttcgcgactggctgaactggtggggaggttgggcagtcggagagtattgggacacgaacgttgacgctttgctttcctgggcttacgagagcggtgcaaaggtctttgactttccactttattataagatggacgaggcttttgataacaataatattcctgcattagtatacgcgttacaaaacggtcagacggttgtaagcagagatccattcaaggcagttacatttgtcgccaaccatgatacggacatcatttggaacaagtaccctgcctacgcatttatcttaacttacgagggtcaaccagtaattttctatagagattttgaagaatggttgaacaaggataaacttattaacctcatctggatacacgaccacctggctggtgggtccacgacaatagtctactacgacaacgatgagttaatctttgttcggaacggagattcccgccgcccaggtttaatcacttacataaatctgtcccctaactgggtggggcgttgggtgtatgtcccgaaatttgcaggtgcctgtatacatgagtatactggcaatcttggaggctgggtagataaacgtgttgacagtagtggatgggtttacttggaggctccgccgcatgatcctgccaacgggtactatggatattccgtgtggtcatattgcggggtagggtaa

SEQ ID NO: 29 - DNA, 1551 bp, synthetic construct
  source 1..1551: mol_type = other DNA
atgagaaaaaagattacgttagcatgcaagacatgcggaaaccgtaattatacgacaatgaagagctctgcatcagcggcggccggtgcacatatggagctcatggcagccccgttcaatggcaccatgatgcagtacttcgagtggtacttaccggacgacggtacactgtggaccaaagttgccaacgaggcaaacaacctgagcagcctgggcatcaccgccttatggctgcctccggcctataaaggcaccagtcgtagtgacgtgggctatggcgtgtacgacctgtatgacttaggcgagttcaatcaaaagggcacagtgcgcaccaagtacggcaccgaggcccagtacctgcaagccattcaagccgcccacgccgcaggcatgcaagtgtatgccgatgtggtgttcgatcacaagggcggcgccgatggtacagaatgggtggatgccgtggaggtgaacccgagcgaccgcaaccaggagattagcggcacctaccagatccaggcctggaccaaattcgacttcccgggccgcggcaacacatacagcagcttcaagtggcgctggtaccactttgatggcgtggattgggacgaaagccgtaagctgagccgcatctacaagtttcgtggcaaggcctgggactgggaggtggataccgagttcggtaactacgattacttaatgtatgcagatctggacatggaccacccggaggtggtgaccgagttaaagaactggggtaaatggtatgttaacaccaccaatattgacggcttccgcctggacgccgttaagcatattaagttcagcttcttccctgattggctgagctacgtgcgtagtcagacaggcaagcctctgtttaccgtgggcgagtactggagctacgacattaacaagctgcataactacatcaccaagaccgacggcacaatgagcctgtttgatgcccctctgcacaacaagttctacaccgccagcaagagtggtggtgcctttgacatgcgcaccctgatgaccaacaccctgatgaaagatcagccgaccctggccgtgaccttcgttgacaaccatgataccgagccggtgcaggcattacagagctgggtggacccttggtttaagcctctggcctacgccttcattctgacccgccaagagggttacccgtgtgtgttctacggtgactactacggtattccgcaatataacattcctagcctgaagagcaagatcgacccgctgctgatcgcccgtcgtgactatgcctacggtacacagcacgactatctggaccacagagacatcattggctggacccgtgagggtggcaccgagaagcctggcagcggtttagcagcactgatcaccgacggccctggtggcagcaaatggatgtacgtgggcaagcagcatgccggcaaggtgttctatgacctgacaggcaatcgcagcgataccgtgaccatcaacagtgacggctggggcgaattcaaggttaatggtggtagcgtgagtgtgtgggttccgcgctaa

SEQ ID NO: 30 - DNA, 1842 bp, synthetic construct
  source 1..1842: mol_type = other DNA
atgtttgcaaaacgattcaaaacctctttactgccgttattcgctggatttttattgctgtttcatttggttctggcaggaccggcggctgcgagtgctgaagccatggcaagcccgctgaccaaaccgagcaccccgattgcagcaaccaatattcagaaaagcgcagattttccgatttggtggaaacaggcagtcttctatcagatatatccgcgtagctttaaagatagcaatggggatggtattggggatattccgggtattattgaaaaactggattatctgaaaatgctgggtgttgatgcaatttggattaatccgcattatgaaagcccgaataccgataatggttatgatattagcgattatcgaaagattatgaaagaatatggtagcatggcagattttgatcgcctggttgcagagatgaacaagcgcggtatgcgcctgatgattgatattgttattaatcataccagcgatcgccatcgttggtttgttcagagccgtagcggtaaagataatccgtatcgcgattattatttttggcgtgatggtaaacagggtcaggcaccgaacaattatccgagcttctttggtggtagcgcatggcagctggataaacagaccgatcagtattatctgcattattttgcaccgcagcagccggatctgaattgggataatccgaaagttcgtgcagaactgtatgatattctgcgcttttggctggataaaggtgttagcggtctgcgctttgataccgttgcaacctttagcaaaattccgggttttccggatctgagcaaagcacagctgaaaaattttgcagaagcatataccgaaggtccgaatattcataaatatattcatgagatgaatcgccaggttctgagcaaatataatgttgcaaccgcaggtgaaatttttggtgttccggttagcgcaatgccggattattttgatcgtcgtcgcgaagaactgaatattgcattcacctttgatctgattcgcctggatcgctatccggatcagcgttggcgtcgtaaaccgtggaccctgagccagtttcgtcaggttattagccagaccgatcgtgcagcaggtgaatttggttggaatgcattcttcctggataatcatgataatccgcgtcaggttagccattttggagacgatagcccgcagtggcgcgaacgtagcgcaaaagcactggcaaccctgctgctgacccagcgcgcaaccccgtttatttttcagggtgcagaactgggtatgaccaattatccgtttaaaaatattgaagaatttgatgatattgaagttaaaggtttttggaatgattatgttgcaagcggtaaagttaatgcagcagaatttcttcaagaagttcgtatgaccagccgcgataatagccgtaccccgatgcagtggaatgatagcgttaatgcaggttttacccagggtaaaccgtggtttcatctgaatccgaattataagcagattaatgcagcacgcgaagttaacaaaccggatagcgtttttagctattatcgccagctgattaatctgcgtcatcagattccggcactgaccagcggtgaatatcgcgatttggacccgcagaacaaccaggtttatgcatatacccgcattctggataatgagaaatatctggttgttgttaattttaaaccggaacagctgcattatgcactgccggataatctgaccattgcaagcagcctgctggaaaatgttcatcagccgagcctacaggaaaatgcaagcaccctgaccctggcaccgtggcaggcaggtatatataagctgaattaa

SEQ ID NO: 31 - DNA, 327 bp, synthetic construct
  source 1..327: mol_type = other DNA
ggcggcgttctgtttctgcttcggtatgtgattgtgaagctggcttacagaagagcggtaaaagaagaaataaaaaagaaatcatcttttttgtttggaaagcgagggaagcgttcacagtttcgggcagctttttttataggaacattgatttgtattcactctgccaagttgttttgatagagtgattgtgataattttaatgtaagcgataacaaaattctccagtcttcacatcggtttgaaaggaggaagcggaagaatgaagtaagagggatttttgactccgaagtaagtcttcaaaaaatcaaataaggagtgtcaaga

SEQ ID NO: 32 - DNA, 280 bp, synthetic construct
  source 1..280: mol_type = other DNA
ttctcaaaaaatactacctgtcccttgctgatttttaaacgagcacgagagcaaaacccccctttgctgaggtggcagagggcaggtttttttgtttcttttttctcgtaaaaaaaagaaaggtcttaaaggttttatggttttggtcggcactgccgacagcctcgcagagcacacactttatgaatataaagtatagtgtgttatactttacttggaagtggttgccggaaagagcgaaaatgcctcacatttgtgccacctaaaaaggagcgattta

SEQ ID NO: 33 - DNA, 361 bp, synthetic construct
  source 1..361: mol_type = other DNA
ggcatgtatccgaatcgtacaaaagaaccttttcataagaattggaagggcgtatattcacttaaaattcacagttggtgagactttaagattacaaaaaaggtaaaaaaaccaaatctctcagacataaggcaaatgagaaatttcccgctctatgggaaaaaacactaaagttgatcaaatgacctaagtgcgccaaacgtgttacgggacgagctatctcatggtataaatggaattgtaaacgttatcaaggaggtcgtcatatgaagaaaaaatcattctcaatcgtaatagcgggcggagggagcactttcactccaggatcttaaataaataaatttatcaaggaggtgaattc

SEQ ID NO: 34 - protein, 649 aa, synthetic construct
MKTFIKERGLAFFLIAVVLLWIKTYVGYVLNFNLGIDNTIQKILLFVNPLSSSLFFLGFGLLFKKKLQQTAIIVIHFLMSFLLYANIVYYRFFNDFITIPVIMQAKTNGGQLGDSAFSLMRPTDAFYFIDTIILIILAIKVNKPAETSSKKSFRIIFASSILVFLINLAVAESDRPELLTRSFDRNYLVKYLGTYNFTIYDAVQNIKSNSQRALADSSDVTEVENYMKANYDVPNNVYFGKAEGKNVIYVSLESLQSFIIDYKIDGKEVTPFLNKLAHDNETFYFDNFFHQTGQGKTSDAEFMMENSLYPLAQGSVFVNKAQNTLQSVPAILKSKNYTSATFHGNTQTFWNRNEMYKAEGIDKFFDSAYYDMNEENTKNYGMKDKPFFKESMPLLESLPQPFYTKFITLSNHFPFGMDEGDTDFPAGDFGDSVVDNYFQSAHYLDQSIEQFFNDLKKDGLYDKSIIVMYGDHYGISENHNKAMAKVLGKDEITDYDNAQLQRVPLFIHAAGVKGEKVHKYAGDVDVAPTILHLLGVDTKDYLMSGSDILSKEHREVIPFRNGDFISPKYTKISGKYYDTKTGKELDESEVDKSEDSLVKKELEMSDKIINGDLLRFYEPKGFKKVNPSDYDYTKHDEDSSETSKDNEDK

SEQ ID NO: 35 - DNA, 1835 bp, synthetic construct
  source 1..1835: mol_type = other DNA
atgaaaacatttataaaagaaagaggactggccttcttcttaattgcggtcgtcctgttatggatcaaaacgtatgtcggttatgtcctgaatttcaacttaggaatagacaacacgatacaaaaaatattgctttttgtgaatcctcttagctcaagcttgttctttcttggctttggactcttgttcaagaaaaaattacagcagacagccattatagtgattcattttttaatgtcttttttactgtacgccaacattgtgtactacagatttttcaatgattttattacaattccggtcattatgcaggctaaaacaaacggcggccaactcggtgacagcgcattttcgctgatgagaccgactgacgccttttactttatcgatacgatcatcctgatcatcttggcgatcaaagtaaacaagcctgccgaaacgtcaagcaaaaaatcgttccgaattatttttgcgtcttcaattcttgtgttcttgatcaacctggcagttgcggaatcagaccgtcctgaattgctgacaagatcattcgaccggaactatcttgtgaaatacttgggaacatacaatttcacgatttatgacgctgtacagaatatcaagtccaacagccagcgcgcgcttgccgattccagcgacgtaacggaagtagaaaactacatgaaagccaattacgatgtgccgaataacgtgtatttcggcaaagcggaaggaaaaaacgtcatttacgtttcacttgaatctttgcagtcatttatcatcgactataaaattgacggcaaagaagtgacaccattcttaaataaactggcacatgataacgaaacgttctactttgataactttttccaccaaacgggacaaggtaaaacatctgatgctgaatttatgatggaaaactctctgtacccgctggctcaaggttcagttttcgtaaacaaagcgcaaaacacgctgcaatccgttccggcgattctgaagtctaagaattacacatctgctactttccacgggaacacgcagacgttctggaaccgtaacgaaatgtacaaggcggaaggcattgataaattctttgattctgcttactatgacatgaacgaagaaaacacgaaaaactacggcatgaaagacaaaccgttcttcaaagaatcaatgccgctgctggaaagcctgccgcagccgttctatacgaagttcattaccctttccaaccacttcccattcggaatggatgagggggatacagacttcccggctggagactttggtgactctgtcgtcgataactatttccagtcagcccattaccttgatcagtccattgaacaattcttcaatgatctgaaaaaagacgggttatatgataaatcgattattgtgatgtacggagaccactacggcatctctgaaaaccacaataaagcgatggcgaaagtgcttggcaaggatgaaatcactgattacgacaacgcccagcttcaacgggtgccgctctttatccacgctgccggcgtgaagggcgagaaagttcataaatatgccggagacgttgatgtggctcctaccattctgcatctgctcggagtggatacgaaggactatctgatgtccggttctgatattttatcgaaagaacaccgtgaagtgattccgttccgaaacggagactttatttcaccgaagtacacgaaaatatccggtaagtattacgacacgaaaaccggaaaagaactcgatgaatccgaagtcgacaagtcagaagactcactcgtcaagaaggaacttgaaatgtccgataaaatcataaacggaga

(19) United States
(12) Patent Application Publication
(10) Pub. No.: US 2025/0034539 A1
(43) Pub. Date: Jan. 30, 2025

(54) Method for Increasing Expression Level of Recombinant Proteins in Bacillus Subtilis Through Co-expressing Bacillus-derived Enhancer Factor

(71) Applicant: Jiangnan University, Wuxi (CN)

(72) Inventors: Kang ZHANG, Wuxi (CN); Jing WU, Wuxi (CN); Xuyang ZHU, Wuxi (CN); Hui LUO, Wuxi (CN)

(21) Appl. No.: 18/919,792

(22) Filed: Oct. 18, 2024

Related U.S. Application Data
(63) Continuation of application No. PCT/CN2023/135786, filed on Dec. 1, 2023.

(30) Foreign Application Priority Data
May 23, 2023 (CN) 202310581833.0

(51) Int. Cl. C12N 9/28; C12N 1/20; C12N 9/90; C12N 15/75

(52) U.S. Cl. CPC C12N 9/2417; C12N 1/20; C12N 9/90; C12N 15/75; C12Y 302/01001; C12Y 504/99011

(57) ABSTRACT

Disclosed is a method for increasing expression level of recombinant proteins in Bacillus subtilis through co-expressing Bacillus-derived enhancer factor, belonging to the technical field of enzyme engineering. According to the present disclosure, genes encoding recombinant proteins are respectively co-expressed with Bacillus-derived PonA, PonA truncated forms, OppA or SppA, so as to improve the expression level of the recombinant proteins. The results show that the method provided by the present disclosure can increase the expression level of ultra-high temperature amylase, medium temperature amylase or sucrose isomerase at the shake flask level to 3.96 times, 1.49 times and 2.26 times. The expression level of ultra-high temperature amylase can be increased by 26% in high-density fermentation of a 3L bioreactor. The B. subtilis provides a new developable target as an recombinant protein expression host, and provides technical support for the optimization and modification of the enhancer factors.

### REFERENCE TO SEQUENCE LISTING

The instant application contains a Sequence Listing in XML format as a file named “YGHY-2024-04.xml”, created on Oct. 15, 2024, of 77056 bytes in size, and which is hereby incorporated by reference in its entirety.

### TECHNICAL FIELD

The present disclosure relates to a method for increasing expression level of recombinant proteins in Bacillus subtilis through co-expressing Bacillus-derived enhancer factor, belonging to the technical field of enzyme engineering.

### BACKGROUND

B. subtilis is a Gram-positive strain of Bacillus Cohn. B. subtilis is listed as a strain certified by the GRAS (Generally Recognized as Safe) list of the US FDA because it does not produce toxins and thermosensitive proteins. Its characteristics of easy isolation and cultivation, high-density fermentation, and strong protein secretion ability make B. subtilis, as an important industrial strain, used to produce a variety of exogenous recombinant proteins and metabolites, and its excellent traits have higher identification value. Furthermore, B. subtilis has a clear genetic background and mature molecular biology gene editing methods, which is conducive to performing genetic modification on B. subtilis to enhance the expression ability of B. subtilis as a host for recombinant protein production.

Further improving the exogenous protein production capacity of B. subtilis is a top priority in current research. Currently, many strategies have been adopted to modify this model microorganism to obtain higher recombinant protein yield, such as overexpressing Sec pathway-related factors, knocking out intracellular or extracellular proteases, and increasing chaperone protein content. However, the selection of these modified targets largely depends on the existing annotations of functional genes in B. subtilis, while the synthesis and secretion of proteins are not regulated by a single pathway. Some studies have pointed out that genes that regulate physiologically related functions play an important role in a process of protein expression or metabolite accumulation. For example, Jennifer Staudacher increased the expression level of recombinant proteins to 3 times by up-regulating the expression level of translation initiation factors, and Xiao-Ran Jiang increased PHB production to 1.8 times by regulating cell morphology control genes. Therefore, the current annotation of protein synthesis and secretion related pathways is incomplete, which means that current modification strategies are relatively one-sided, consequently limiting the further development of B. subtilis as an expression host.

At present, the identification of key factors involved in protein expression is still incomplete, so there is an urgent need to develop other novel factors that can enhance the expression of recombinant proteins in B. subtilis based on existing basis.

### SUMMARY

In order to solve the problem of limited expression of recombinant proteins in B. subtilis in the prior art, the present disclosure provides a method for increasing expression level of recombinant proteins in B. subtilis. The method involves overexpressing class A penicillin-binding proteins PonA derived from Bacillus, truncated forms of the class A penicillin-binding proteins PonA, an ABC pathway transporter OppA or a signal peptide peptidase SppA.

In one embodiment, the PonA refers to class A penicillin-binding proteins derived from B. subtilis, Bacillus licheniformis, Bacillus amyloliquefaciens, Bacillus megaterium, or Geobacillus stearothermophilus.

In one embodiment, the protein PonA derived from B. subtilis is PonA (Bs) with an amino acid sequence as shown in SEQ ID NO.1.

In one embodiment, the protein PonA derived from B. licheniformis is PonA (BI) with an amino acid sequence as shown in SEQ ID NO.2.

In one embodiment, the protein PonA derived from B. amyloliquefaciens is PonA (Ba) with an amino acid sequence as shown in SEQ ID NO.3.

In one embodiment, the protein PonA derived from B. megaterium is PonA (Bm) with an amino acid sequence as shown in SEQ ID NO.4.

In one embodiment, the protein PonA derived from G. stearothermophilus is PonA (Gs) with an amino acid sequence as shown in SEQ ID NO.5.

In one embodiment, the truncated forms of the PonA refer to truncated forms of class A penicillin-binding proteins derived from B. subtilis, B. licheniformis, B. amyloliquefaciens, B. megaterium, or G. stearothermophilus.

In one embodiment, the truncated form of the protein PonA derived from B. subtilis is DcPonA (Bs) with an amino acid sequence as shown in SEQ ID NO.6.

In one embodiment, the truncated form of the protein PonA derived from B. licheniformis is DcPonA (BI) with an amino acid sequence as shown in SEQ ID NO.7.

In one embodiment, the truncated form of the protein PonA derived from B. amyloliquefaciens is DcPonA (Ba) with an amino acid sequence as shown in SEQ ID NO.8.

In one embodiment, the truncated form of the protein PonA derived from B. megaterium is DcPonA (Bm) with an amino acid sequence as shown in SEQ ID NO.9.

In one embodiment, the truncated form of the protein PonA derived from G. stearothermophilus is DcPonA (Gs) with an amino acid sequence as shown in SEQ ID NO.10.

In one embodiment, an ABC pathway transporter OppA derived from B. subtilis has an amino acid sequence as shown in SEQ ID NO.21; and a signal peptide peptidase SppA derived from B. subtilis has an amino acid sequence as shown in SEQ ID NO.22.

In one embodiment, the recombinant proteins include, but are not limited to, ultra-high temperature amylase, medium temperature amylase, or sucrose isomerase.

In one embodiment, the ultra-high temperature amylase has an amino acid sequence as shown in SEQ ID NO.23.

In one embodiment, the medium temperature amylase has an amino acid sequence as shown in SEQ ID NO.24.

In one embodiment, the sucrose isomerase has an amino acid sequence as shown in SEQ ID NO.25.

In one embodiment, pUB110 is used as an expression vector to express ultra-high temperature amylase, medium temperature amylase or sucrose isomerase.

In one embodiment, pAD123 is used as an expression vector to express the class A penicillin-binding proteins PonA, the truncated forms of the class A penicillin-binding proteins PonA, the ABC pathway transporter OppA or the signal peptide peptidase SppA.

In one embodiment, the method involves expressing recombinant proteins as well as the PonA, OppA, and SppA in the same host.

In one embodiment, the host includes, but is not limited to, B. subtilis SCK6. In one embodiment, the recombinant proteins are expressed through a constitutive

promoter P_{amyQ′}, and a nucleotide sequence of the promoter P_{amyQ′} is shown in SEQ ID NO.31.

In one embodiment, the class A penicillin-binding protein PonA, the truncated form of the class A penicillin-binding protein PonA, the ABC pathway transporter OppA or the signal peptide peptidase SppA is expressed through a promoter P_{glv} or a promoter P_{HpaII}.

In one embodiment, a nucleotide sequence of the promoter P_{HpaII} is shown in SEQ ID NO.32.

In one embodiment, a nucleotide sequence of the promoter P_{glv} is shown in SEQ ID NO.33.

The present disclosure further provides a recombinant strain of B. subtilis with increased expression level of recombinant proteins. The recombinant strain of B. subtilis respectively co-expresses the Bacillus-derived class A penicillin-binding proteins PonA or DcPonA, the B. subtilis-derived ABC pathway transporter OppA or the B. subtilis-derived signal peptide peptidase SppA, while expressing the recombinant proteins.

In one embodiment, the exogenous protein refers to a protein that is exogenous with respect to B. subtilis capable of expressing and secreting the protein. For example, the exogenous protein may be a microorganism-derived protein, a plant-derived protein, an animal-derived protein, a virus-derived protein, or even a protein whose amino acid sequence is artificially designed. In particular, the heterologous protein may be a human-derived protein. The heterologous protein may be a monomeric protein or a multimeric protein. The “multimeric protein” refers to a protein that can exist as a multimer including two or more subunits. In the multimer, the subunits may be linked by covalent bonds, such as a disulfide bonds, or by non-covalent bonds, such as interactions between hydrogen bonds and hydrophobicity, or by a combination thereof. Preferably, one or more intermolecular disulfide bonds are included in the multimer. The multimer may be a homomultimer including a single type of subunits or a heteromultimer including two or more types of subunits. It should be noted that in the case where the multimeric protein is a heteromultimer, at least one of the subunits constituting the multimer needs to be a heterologous protein. That is, all subunits may be heterologous, or only a portion of the subunits may be heterologous. The heterologous protein may be a natural secretory protein or a natural non-secretory protein; and preferably, it is a natural secretory protein. In addition, the heterologous protein may be a natural Tat-dependent secretory protein or a natural Sec-dependent secretory protein.

In one embodiment, pUB110 is used as an expression vector to express ultra-high temperature amylase, medium temperature amylase or sucrose isomerase.

In one embodiment, pAD123 is used as an expression vector to express the class A penicillin-binding proteins PonA, the truncated forms of the class A penicillin-binding proteins PonA, the ABC pathway transporter OppA or the signal peptide peptidase SppA.

The present disclosure further provides a method for constructing a recombinant strain of B. subtilis, which includes introducing genes encoding recombinant proteins and genes encoding enhancer factors into B. subtilis.

In one embodiment, the genes encoding the enhancer factors include, but are not limited to, the genes encoding the class A penicillin-binding proteins PonA, the truncated forms of the class A penicillin-binding proteins PonA, the ABC pathway transporter OppA or the signal peptide peptidase SppA.

In one embodiment, the genes encoding the class A penicillin-binding proteins PonA have nucleotide sequences as shown in any one of SEQ ID NO.11 to SEQ ID NO.15.

In one embodiment, the genes encoding the truncated forms of the class A penicillin-binding proteins PonA have nucleotide sequences as shown in any one of SEQ ID NO.16 to SEQ ID NO.20.

In one embodiment, the genes encoding the recombinant proteins include, but are not limited to, genes encoding ultra-high temperature amylase, medium temperature amylase or sucrose isomerase.

In one embodiment, the gene encoding the ultra-high temperature amylase has a nucleotide sequence as shown in SEQ ID NO.28.

In one embodiment, the gene encoding the medium temperature amylase has a nucleotide sequence as shown in SEQ ID NO.29.

In one embodiment, gene encoding the sucrose isomerase has a nucleotide sequence as shown in SEQ ID NO.30.

The present disclosure further provides application of the recombinant strain of B. subtilis in production of ultra-high temperature amylase, medium temperature amylase or sucrose isomerase.

In one embodiment, the application includes culturing the recombinant strain in a fermentation medium for a period of time to collect ultra-high temperature amylase, medium temperature amylase or sucrose isomerase.

In one embodiment, the application is fermentation at 30-40° C., or 30-37° C., or 35-40° C., or 37-40° C., or 35-37° C.

In one embodiment, the fermentation lasts for at least 24 h, or at least 36 h, or at least 42 h, or at least 48 h, or at least 60 h.

In one embodiment, the fermentation medium contains yeast powder, soy peptone, ammonium citrate, and metal ions.

In one embodiment, the fermentation medium contains 9.0 g/L yeast powder, 18 g/L soy peptone, 1.0 g/L ammonium citrate, 14.66 g/L K₂HPO₄·3H₂O, 2.68 g/L (NH₄)₂SO₄, 2.0 g/L Na₂SO₃, 3.49 g/L NaH₂PO₄, 0.49 g/L MgSO₄, and 0.3% (v/v) TES metal ion liquid.

In one embodiment, the TES metal ion liquid contains: 0.5 g/L CaCl₂, 0.18 g/L ZnSO₄·7H₂O, 0.1 g/L MnSO₄·H₂O, 8.35 g/L FeCl₃, 0.16 g/L CuSO₄·5H₂O, 0.18 g/L CoCl₂·6H₂O, and 10.5 g/L Na₂·EDTA.

In one embodiment, feeding is further performed during the fermentation.

In one embodiment, the feeding is to supplement a glucose-containing feed medium.

In one embodiment, the feed medium contains glucose, soy peptone, yeast powder, and metal ions.

In one embodiment, the feed medium contains 250.0 g/L glucose, 66.66 g/L soy peptone, 33.33 g/L yeast powder, 0.39 g/L MgSO₄, and 4.0% (v/v) TES metal ion liquid.

The present disclosure further provides applications of the recombinant strain of B. subtilis or the method in the production of products in the fields such as food, chemical industry, sugar refining, and feed.

Beneficial effects:
(1) The present disclosure significantly increases the expression level of recombinant proteins in B. subtilis by overexpressing the Bacillus-derived enhancer factor PonA, OppA, or SppA, which can increase the enzyme activity of the ultra-high temperature amylase to 2.03 times, 1.42 times, and 1.92 times that of the wild-type amylase.
(2) The present disclosure also screens proteins PonA of the enhancer factors from different sources, making the expression level of the co-expressed ultra-high temperature amylase increased to 1.71 to 3.96 times compared to control strains.
(3) The present disclosure truncates the FN3 domains of the proteins PonA from different sources, and the co-expression of the obtained truncated forms DcPonA and the ultra-high temperature amylase can increase the expression level of the ultra-high temperature amylase by 2.02 to 3.35 times compared to control strains.

### BRIEF DESCRIPTION OF FIGURES

FIG. 1 shows schematic diagrams illustrating effects of a recombinant strain co-expressing PonA, OppA, or SppA derived from B. subtilis on the enzyme activities of ultra-high temperature amylase, medium temperature amylase and sucrose isomerase.

FIG. 2 is a schematic diagram showing a shake flask cell concentration and enzyme activity of a recombinant strain co-expressing Bacillus-derived PonA or its genetically engineered truncated forms DcPonA and ultra-high temperature amylase.

FIG. 3 shows cell concentration and enzyme activity curves of a recombinant strain co-expressing PonA (Bs) and ultra-high temperature amylase in a 3 L bioreactor; (a) SCK6/pfa-ck, (b) SCK6/pfa-ponA (Bs).

FIG. 4 is an SDS-PAGE analysis electrophoretogram of samples fermented in a 3 L bioreactor by a recombinant strain co-expressing PonA (Bs) and ultra-high temperature amylase.

FIG. 5 is a schematic diagram showing a shake flask cell concentration and enzyme activity of a recombinant strain co-expressing B. subtilis-derived PonA or its homogeneous protein teichoicase LtaS and ultra-high temperature amylase.

### DETAILED DESCRIPTION

### (I) The Abbreviations and Full Names Involved in the Present Disclosure:

PonA: Class A penicillin-binding proteins.

OppA: ABC pathway transporter.

SppA: Signal peptide peptidase.

PonA (Bs): Class A penicillin-binding protein derived from B. subtilis 168.

PonA (BI): Class A penicillin-binding protein derived from B. licheniformis DSM13.

PonA (Ba): Class A penicillin-binding protein derived from B. amyloliquefaciens DSM7.

PonA (Bm): Class A penicillin-binding protein derived from B. megaterium DSM319.

PonA (Gs): Class A penicillin-binding protein derived from G. stearothermophilus ATCC 7953.

DcPonA (Bs): Truncated form of class A penicillin-binding protein derived from B. subtilis 168.

DcPonA (BI): Truncated form of class A penicillin-binding protein derived from B. licheniformis DSM13.

DcPonA (Ba): Truncated form of class A penicillin-binding protein derived from B. amyloliquefaciens DSM7.

DcPonA (Bm): Truncated form of class A penicillin-binding protein derived from B. megaterium DSM319.

DcPonA (Gs): Truncated form of class A penicillin-binding protein derived from G. stearothermophilus ATCC 7953.

LB: Luria-Bertani medium, used for the culture of B. subtilis.

TB: Terrific Broth, i.e., super broth medium.

### (II) Detection Methods Involved in the Specific Examples of the Present Disclosure:

### (1) Biomass Determination:

Expressed as cell concentration (OD₆₀₀). After sampling, a sample was diluted with deionized water by an appropriate multiple and then placed in a spectrophotometer to measure the absorbance at 600 nm.

### (2) Ultra-High Temperature Amylase Activity Assay:

The specific operation can be found in the literature (Tan Ruiting, Zhang Kang, Wu Jing. Secretory expression of Thermophilic archaea ultra-high temperature α-amylase in B. subtilis [J]. Genomics and Applied Biology). The enzyme activity determination formula is: U (U·mL⁻¹)=n×(6.0539×Δ540+0.3494), where U is the enzyme activity unit, n is the dilution factor of an enzyme solution, and Δ540 is the absorbance value of a sample measured at OD₅₄₀ minus the absorbance value of a blank group.

### (3) Medium Temperature Amylase Activity Assay:

The specific operation can be found in the literature (Yao D, Zhang K, Zhu X, et al. Enhanced extracellular α-amylase production in Brevibacillus choshinensis by optimizing extracellular degradation and folding environment [J]. Journal of Industrial Microbiology and Biotechnology, 2021(1):1.). The enzyme activity determination formula is: U(U·mL⁻¹)=n×(6.0539×Δ540+0.3494), where U is the enzyme activity unit, n is the dilution factor of an enzyme solution, and Δ540 is the absorbance value of a sample measured at OD₅₄₀ minus the absorbance value of a blank group.

### (4) Sucrose Isomerase Activity Assay:

The specific operation can be found in the literature (Liu Juntong, Wu Jing, Chen Sheng. Expression and fermentation optimization of sucrose isomerase from Pantoea dispersa in Escherichia coli [J]. Chinese Journal of Biotechnology, 2016, 032(008):1070-1080). The enzyme activity determination formula is: U (U·mL⁻¹)=n×(6.0539×Δ540+0.3494), where U is the enzyme activity unit, n is the dilution factor of an enzyme solution, and Δ540 is the absorbance value of a sample measured at OD₅₄₀ minus the absorbance value of a blank group.

### (III) Mediums Involved in the Specific Examples of the Present Disclosure:

The mediums were all prepared using ddH₂O, and sterilized at 121° C. for 20 min after preparation.

LB liquid medium: 5.0 g/L yeast powder, 10.0 g/L tryptone, 10.0 g/L NaCl.

TB liquid medium: 24.0 g/L yeast powder, 12.0 g/L tryptone, 5.0 g/L glycerol, 16.43 g/L K₂HPO₄·3H₂O, 2.31 g/L KH₂PO₄.

3 L fermentation tank basic culture medium: 9.0 g/L yeast powder, 18 g/L soy peptone, 1.0 g/L ammonium citrate, 14.66 g/L K₂HPO₄·3H₂O, 2.68 g/L (NH₄) 2SO₄, 2.0 g/L Na₂SO₃, 3.49 g/L NaH₂PO₄, 0.49 g/L MgSO₄, 0.3% (v/v) TES metal ion liquid.

3 L fermentation tank feed medium: 250.0 g/L glucose, 66.66 g/L soy peptone, 33.33 g/L yeast powder, 0.39 g/L MgSO₄, 4.0% (v/v) TES metal ion liquid.

TES metal ionic liquid: 0.5 g/L CaCl₂), 0.18 g/L ZnSO₄·7H₂O, 0.1 g/L MnSO₄·H₂O, 8.35 g/L FeCl₃, 0.16 g/L CuSO₄·5H₂O, 0.18 g/L CoCl₂·6H₂O, 10.5 g/L Na₂·EDTA.

### Example 1 Construction of Recombinant Strain Expressing Ultra-High Temperature Amylase, Medium Temperature Amylase, or Sucrose Isomerase

Gene fragments pfa, amyS, and si with nucleotide sequences as shown in SEQ ID NO.28, SEQ ID NO.29, and SEQ ID NO.30 were amplified from strains expressing ultra-high temperature amylase, medium temperature amylase, and sucrose isomerase by using a PCR method. After gel extraction, they were ligated to a pUB110 vector by using a poe-pcr method, and the expression of recombinant proteins was regulated by a constitutive promoter P_{amyQ′} as shown in SEQ ID NO.31. Subsequently, the poe-pcr product was transformed into B. subtilis SCK6, and placed in an ice bath for 20 min and then in a water bath at 37° C. for 20 min; resuscitation was performed at 37° C. and 200 rpm for 3 h; the product was coated on an LB plate containing kanamycin resistance (40 g/mL), and cultured overnight; and after screening verification, recombinant strains were obtained, which were named BSP, BSA, and BSS, respectively. The recombinant strain used to obtain the genes pfa, amyS, and si were constructed by the inventor's team in the early stage (published in: Tan Ruiting, Zhang Kang, Wu Jing. Secretory expression of Thermophilic archaea ultra-high temperature α-amylase in B. subtilis [J]. Genomics and Applied Biology; Yao D, Zhang K, Zhu X, et al. Enhanced extracellular α-amylase production in Brevibacillus choshinensis by optimizing extracellular degradation and folding environment [J]. Journal of Industrial Microbiology and Biotechnology, 2021(1):1.; Liu Juntong, Wu Jing, Chen Sheng. Expression and fermentation optimization of sucrose isomerase from Pantoea dispersa in Escherichia coli [J]. Chinese Journal of Biotechnology, 2016, 032(008):1070-1080).

### Example 2 Construction of Co-Expression Plasmids of Enhancer Factors

(1) Construction of Recombinant Plasmid pAD123-PonA

A gene fragment ponA as shown in SEQ ID NO.11 was amplified from the genome of B. subtilis by using a PCR method. After gel extraction, the gene fragment ponA was ligated to a pAD123 vector by a ClonExpress II one step cloning kit, its expression was regulated by a maltose inducible promoter P_{glv} as shown in SEQ ID NO.33, and then it was transformed into E. coli JM109. The product was placed in an ice bath for 30 min, in a water bath at 42° C. for 90 s and then in the ice bath for 3 min; resuscitation was performed at 37° C. and 200 rpm for 1 h; after that, the product was coated on an LB plate containing ampicillin resistance (100 μg/mL), and cultured overnight; and after screening verification, a recombinant plasmid was obtained.

(2) Construction of Recombinant Plasmid pAD123-OppA

A gene fragment oppA with a nucleotide sequence as shown in SEQ ID NO.26 was amplified from the genome of B. subtilis by using a PCR method. After gel extraction, the gene fragment oppA was ligated to a pAD123 vector by a ClonExpress II one step cloning kit, its expression was regulated by a maltose inducible promoter P_{glv} as shown in SEQ ID NO.33, and then it was transformed into E. coli JM109. The product was placed in an ice bath for 30 min, in a water bath at 42° C. for 90 s and then in the ice bath for 3 min; resuscitation was performed at 37° C. and 200 rpm for 1 h; after that, the product was coated on an LB plate containing ampicillin resistance (100 μg/mL), and cultured overnight; and after screening verification, a recombinant plasmid was obtained.

(3) Construction of Recombinant Plasmid pAD123-SppA

A gene fragment sppA with the sequence as shown in SEQ ID NO.22 was amplified from the genome of B. subtilis 168 by using a PCR method. After gel extraction, the gene fragment sppA was ligated to a pAD123 vector by a ClonExpress II one step cloning kit, its expression was regulated by a maltose inducible promoter P_{glv} as shown in SEQ ID NO.33, and then it was transformed into E. coli JM109. The product was placed in an ice bath for 30 min, in a water bath at 42° C. for 90 s and then in the ice bath for 3 min; resuscitation was performed at 37° C. and 200 rpm for 1 h; after that, the product was coated on an LB plate containing ampicillin resistance (100 μg/mL), and cultured overnight; and after screening verification, a recombinant plasmid was obtained.

### Example 3 Construction of Co-Expression Recombinant Strain Producing Ultra-High Temperature Amylase, Medium Temperature Amylase or Sucrose Isomerase

The recombinant plasmids pAD123-PonA, pAD123-OppA and pAD123-SppA constructed correctly in Example 2 were transformed into competent cells containing the recombinant strains BSP, BSA and BSS constructed in Example 1, respectively. Each of the products was placed in an ice bath for 20 min and then in a water bath at 37° C. for 20 min; resuscitation was performed at 37° C. and 200 rpm for 3 h; after that, the product was coated on an LB plate containing kanamycin resistance (40 μg/mL) and chloramphenicol resistance (10 μg/mL), and cultured overnight; and recombinant strains capable of expressing both recombinant proteins and enhancer factors were obtained. The recombinant strains proved to be correct by verification were inoculated into a 10 mL LB medium containing kanamycin resistance (40 g/mL) and chloramphenicol resistance (10 g/mL) at an inoculation amount of 2% %, and cultured at 37° C. and 200 rpm for 10 h as fermentation seed solutions. Subsequently, each of the products was transferred at an inoculation amount of 5% into a 50 mL TB medium containing the same concentration of antibiotics, and 5 g/L maltose was added at the same time to induce expression; and the products were first cultured for 2 h at 37° C., and then the temperature was adjusted to 33° C. until the fermentation end point. After the strain capable of expressing ultra-high temperature amylase was cultured for 60 h, and the strains capable of expressing medium temperature amylase and sucrose isomerase were cultured for 48 h, the fermentation supernatant was appropriately diluted, and the enzyme activities were measured.

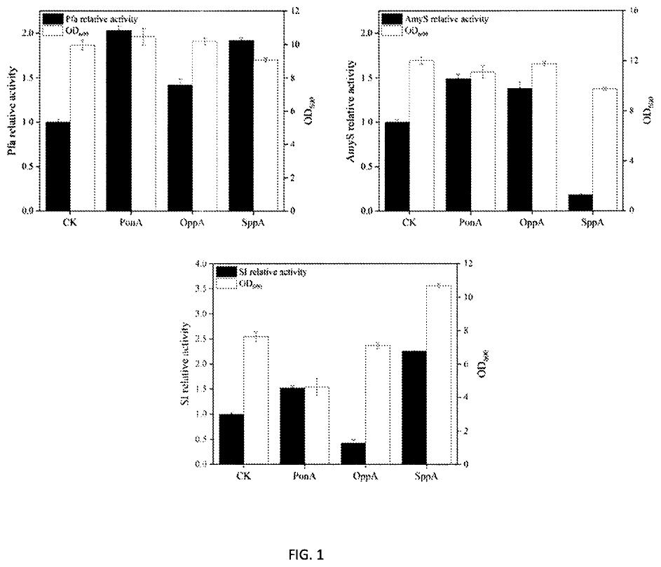

The activities of three recombinant protein in the fermentation supernatant were shown in FIG. 1. When the model enzyme was ultra-high temperature amylase, the co-expression of PonA, OppA, and SppA increased the enzyme activities to 2.03 times, 1.42 times, and 1.92 times that of the wild-type amylase, respectively, with no significant difference in a bacterial cell OD. When the model enzyme was medium temperature amylase, the co-expression of PonA and OppA increased the enzyme activities to 1.49 times and 1.38 times that of the wild-type amylase, respectively, and there was no significant difference in a bacterial cell OD during fermentation of PonA and OppA overexpressing strains. When the model enzyme was sucrose isomerase, the co-expression of PonA and SppA increased the enzyme activities to 1.52 times and 2.26 times that of the wild type amylase, respectively. Overexpression of PonA significantly reduced a bacterial cell OD, while overexpression of SppA significantly increased cell concentration.

### Example 4 Construction of Recombinant Plasmids Expressing PonA from Different Sources

Gene fragments ponA (Bs), ponA (BI), ponA (Ba), ponA (Bm), and ponA (Gs) with nucleotide sequences as shown in SEQ ID NO.11, SEQ ID NO.12, SEQ ID NO.13, SEQ ID NO.14, and SEQ ID NO.15, respectively were amplified from the genomes of B. subtilis 168, B. licheniformis DSM13, B. amyloliquefaciens DSM7, B. megaterium DSM319 and G. stearothermophilus ATCC 7953 by using a PCR method. After gel extraction, the gene fragments were ligated to a pAD123 vector by a ClonExpress II one step cloning kit, their expressions were regulated by a maltose inducible promoter P_{glv}, and then they were transformed into E. coli JM109. Each of the products was placed in an ice bath for 30 min, in a water bath at 42° C. for 90 s and then in the ice bath for 3 min; resuscitation was performed at 37° C. and 200 rpm for 1 h; after that, the products were coated on an LB plate containing ampicillin resistance (100 μg/mL), and cultured overnight; and after screening verification, recombinant plasmids were obtained.

### Example 5 Construction of Recombinant Plasmids Expressing Truncated Forms DcPonA of FN3 Domain

Fragments DcPonA (Bs), DcPonA (BI), DcPonA (Ba), DcPonA (Bm), and DcPonA (Gs) with nucleotide sequences as shown in SEQ ID NO.16, SEQ ID NO.17, SEQ ID NO.18, SEQ ID NO.19, and SEQ ID NO.20, respectively were truncated from the C-terminal FN3 domain of PonA through homologous recombination by using a PCR method. Following the same strategy as in Example 4, the PCR products were ligated to a pAD123 vector by a ClonExpress II one step cloning kit after subjected to gel extraction, and then they were enabled to pass through a dpnI digestion template and transformed into E. coli JM109. Each of the products was placed in an ice bath for 30 min, in a water bath at 42° C. for 90 s and then in the ice bath for 3 min; resuscitation was performed at 37° C. and 200 rpm for 1 h; after that, the products were coated on an LB plate containing ampicillin resistance (100 μg/mL), and cultured overnight; and after screening verification, recombinant plasmids were obtained.

### Example 6 Synthesis of Ultra-High Temperature Amylase with Recombinant Strain Co-Expressing PonA or DcPonA Through Shake Flask Fermentation

The correct recombinant plasmids constructed in Examples 4 and 5 were separately transformed into the recombinant strain BSP constructed in Example 1. Each of the products was placed in an ice bath for 20 min and then in a water bath at 37° C. for 20 min; resuscitation was performed at 37° C. and 200 rpm for 3 h; after that, the product was coated on an LB plate containing kanamycin resistance (40 μg/mL) and chloramphenicol resistance (10 μg/mL), and cultured overnight; and a recombinant strain capable of expressing both ultra-high temperature amylase Pfa and PonA, or a recombinant strain capable of expressing both ultra-high temperature amylase Pfa and DcPonA was obtained. The recombinant strain proved to be correct by verification was inoculated into a 10 mL LB medium containing kanamycin resistance (40 g/mL) and chloramphenicol resistance (10 g/mL) at an inoculation amount of 2% %, and cultured at 37° C. and 200 rpm for 10 h as a fermentation seed solution. Subsequently, the product was transferred at an inoculation amount of 5% into a 50 mL TB medium containing the same concentration of antibiotics, and 5 g/L maltose was added at the same time to induce expression; and the product was first cultured for 2 h at 37° C., and then the temperature was adjusted to 33° C. until culturing for 60 h. Subsequently, the fermentation supernatant was appropriately diluted and the Pfa expression level was measured according to the enzyme activity detection method described above. Without expressing PonA or DcPonA, a recombinant strain obtained by only transforming the plasmid pAD123 to the recombinant strain BSP was used as a control strain.

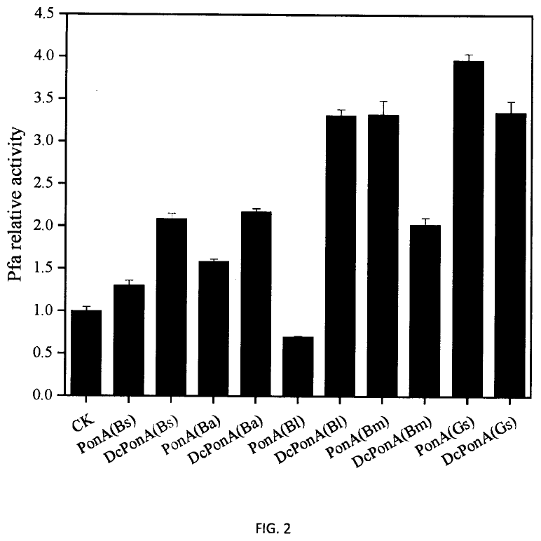

The Pfa enzyme activity in the fermentation supernatant was shown in FIG. 2. Except for PonA (BI), the recombinant strains expressing other Bacillus-derived proteins PonA showed a significant increase in protein expression level compared to the control strain. The enzyme activities of the recombinant strains co-expressing PonA (Bs), PonA (Ba), PonA (Bm) and PonA (Gs) were 74.17 U/mL, 82.07 U/mL, 143.98 U/mL and 171.76 U/mL, respectively after 60 h of fermentation. Compared with the control strain, their enzyme activities were increased to 1.71 times, 1.89 times, 3.32 times and 3.96 times, respectively. The recombinant strain co-expressing DcPonA showed a significant increase in Pfa enzyme activity compared to the control strain. The enzyme activities of the recombinant strains co-expressing DcPonA (Bs), DcPonA (BI), DcPonA (Ba), DcPonA (Bm) and DcPonA (Gs) were 90.48 U/mL, 143.48 U/mL, 94.04 U/mL, 87.68 U/mL and 145.26 U/mL, respectively after 60 h of fermentation. Compared with the control strain, their enzyme activities were increased to 2.09 times, 3.31 times, 2.17 times, 2.02 times, and 3.35 times, respectively.

### Example 7 Promoter Optimization

Construction of a plasmid pAD123-P_{HpaII}-PonA (Bs) for constitutive expression of PonA includes the following specific steps: a promoter P_{HpaII} gene fragment with a nucleotide sequence as shown in SEQ ID NO.32 was amplified from the genome of B. subtilis 168 by using a PCR method. After gel extraction, the gene fragment was ligated to a vector pAD123-PonA (Bs) carrying PonA (Bs) constructed in Example 4 by a ClonExpress II one step cloning kit, and then it was transformed into E. coli JM109. The product was placed in an ice bath for 30 min, in a water bath at 42° C. for 90 s and then in the ice bath for 3 min; resuscitation was performed at 37° C. and 200 rpm for 1 h; after that, the product was coated on an LB plate containing ampicillin resistance (100 μg/mL), and cultured overnight; and after screening verification, the recombinant plasmid pAD123-P_{HpaII}-PonA (Bs) was obtained.

### Example 8 Synthesis of Ultra-High Temperature Amylase by Horizontal Fermentation in 3 L Fermentation Tank

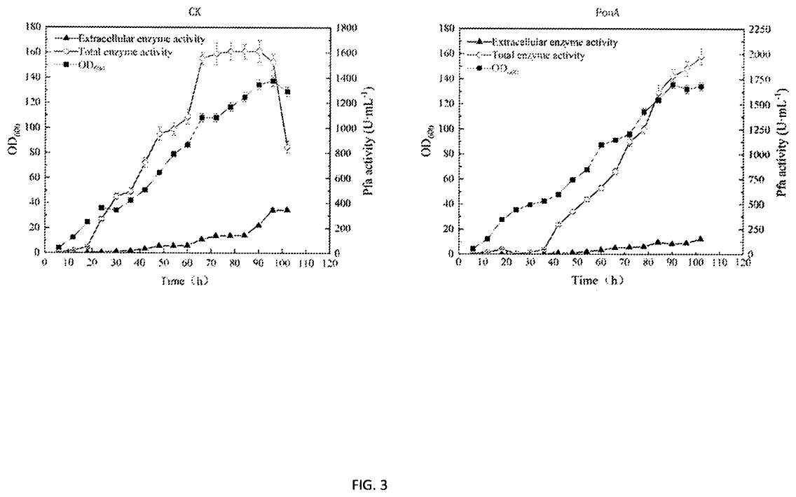

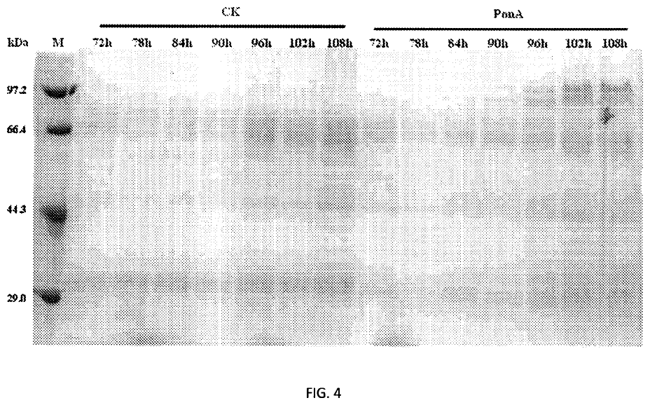

The recombinant plasmid pAD123-P_{HpaII}-PonA (Bs) constructed correctly in Example 7 was transformed into a competent cell constructed in Example 1. The product was placed in an ice bath for 20 min and then in a water bath at 37° C. for 20 min; resuscitation was performed at 37° C. and 200 rpm for 3 h; and after that, the product was coated on an LB plate containing kanamycin resistance (40 μg/mL) and chloramphenicol resistance (10 μg/mL), and cultured overnight, so that a recombinant strain overexpressing constitutive PonA was obtained. The strain freshly deposited in a glycerol tube was cultured at 37° C. for 10 h by plate streaking method, and single colonies were picked into a 50 mL LB medium containing kanamycin (40 μg/mL) and chloramphenicol (10 μg/mL), cultured at 37° C. and 200 rpm for 12 h, and then transferred at a transfer amount of 10% to a 900 mL fermentation medium containing kanamycin (40 g/mL) and chloramphenicol (10 g/mL); the temperature was adjusted to 33° C., the pH was controlled to 7.0 with ammonia water and 20% phosphoric acid, feeding was started when dissolved oxygen rebound occurs after 6.5-8 h of fermentation, and the feeding flow rate was controlled to make the residual sugar content in the system not higher than 0.1 g/L. Timed sampling was performed for the determination of cell concentration and ultra-high temperature amylase activity in the fermentation broth. The total enzyme activity of the control strain reached 1611.86 U/mL (FIG. 3a) at 90 h of fermentation, while the total enzyme activity of the PonA (Bs) co-expression strain reached 2041.73 U/ml at 102 h of fermentation (FIG. 3b), which was 1.26 times higher than that of the control strain. Cell concentrations of the two recombinant strains were basically the same during the high-density fermentation cycle, and the SDS-PAGE showed that protein bands were consistent with the enzyme activity trend (FIG. 4).

### Comparative Example 1

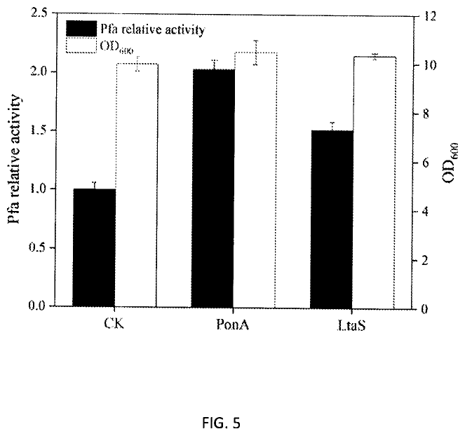

The difference between the specific embodiment and Examples 5-6 lies in the replacement of regulatory factors, specifically: the PonA was replaced with the main teichoicase LtaS in B. subtilis (with the amino acid sequence as shown in SEQ ID NO.34, and the gene sequence as shown in SEQ ID NO.35). The results showed that LtaS overexpression only increased the enzyme activity of Pfa to 1.52 times, which was significantly lower than the improvement effect of the PonA on Pfa expression (2.03 times). Under the same fermentation conditions, there was no significant change in OD₆₀₀ (FIG. 5).

Although the present disclosure has been disclosed as above in exemplary examples, it is not intended to limit the present disclosure. Anyone familiar with this technology can make various changes and modifications without departing from the spirit and scope of the present disclosure. Therefore, the protection scope of the present disclosure shall be as defined in the Claims.

What is claimed is:

1. Recombinant Bacillus subtilis, expressing recombinant proteins, and proteins shown in (a) or (b):
(a) class A penicillin-binding proteins PonA derived from Bacillus;
(b) truncated forms of class A penicillin-binding proteins DcPonA derived from Bacillus;
wherein the recombinant proteins comprise ultra-high temperature amylase, medium temperature amylase or sucrose isomerase;
wherein the proteins PonA having amino acid sequences as shown in any one of SEQ ID NOs:1 to 5; and
wherein the truncated forms of the proteins PonA having amino acid sequences as set forth in any one of SEQ ID NOs:6 to 10.

2. The recombinant B. subtilis according to claim 1, wherein pUB110 is used as an expression vector to express ultra-high temperature amylase, medium temperature amylase or sucrose isomerase.

3. The recombinant B. subtilis according to claim 1, wherein pAD123 is used as an expression vector to express the class A penicillin-binding proteins PonA or the truncated forms of the class A penicillin-binding proteins PonA.

4. The recombinant B. subtilis according to claim 1, wherein the recombinant proteins are expressed through a constitutive promoter P_{amyQ′}.

5. The recombinant B. subtilis according to claim 1, wherein the class A penicillin-binding protein PonA, the truncated form of the class A penicillin-binding protein PonA, an ABC pathway transporter OppA or a signal peptide peptidase SppA is expressed through a promoter P_{glv} or a promoter P_{HpaII}.

6. The recombinant B. subtilis according to claim 1, wherein a host comprises B. subtilis SCK6.

7. A method for increasing expression level of recombinant proteins in B. subtilis, comprising overexpressing class A penicillin-binding proteins PonA derived from Bacillus or truncated forms of class A penicillin-binding proteins PonA while expressing recombinant proteins; and the recombinant proteins comprise ultra-high temperature amylase, medium temperature amylase or sucrose isomerase.

8. The method according to claim 7, wherein pUB110 is used as an expression vector to express ultra-high temperature amylase, medium temperature amylase or sucrose isomerase.

9. The method according to claim 8, wherein pAD123 is used as an expression vector to express the class A penicillin-binding proteins PonA, the truncated forms of the class A penicillin-binding proteins PonA, an ABC pathway transporter OppA or a signal peptide peptidase SppA.

10. A method for preparing enzyme proteins, wherein the recombinant B. subtilis according to claim 1 is fermented in a medium to prepare ultra-high temperature amylase, medium temperature amylase or sucrose isomerase.

11. The method according to claim 10, wherein the method comprises culturing the recombinant strain in a fermentation medium for a period of time to collect ultra-high temperature amylase, medium temperature amylase or sucrose isomerase.

12. The method according to claim 10, wherein feeding is further performed during fermentation.
